(19) United States
(12) Patent Application Publication
(10) Pub. No.: US 2026/0283165 A1
(43) Pub. Date: Sep. 24, 2026

(54) Galactolipase Defined by Ratio of Galactolipase/Phospholipase and/or Galactolipase/Lipase Activity and Its Application in Bakery

(71) Applicant: Puratos N.V., Groot-Bijgaarden (BE)

(72) Inventors: Oksana Shegay, Kozen (BE); Nicolas Guillemin, Andenne (BE); Frédéric Roulling, Verlaine (BE); Fabienne Verté, Destelbergen (BE); Azadeh Mohammadi, Auderghem (BE)

(21) Appl. No.: 19/474,294

(22) PCT Filed: Apr. 11, 2024
(86) PCT No.: PCT/EP2024/059770

§ 371 Date: Oct. 10, 2025

(30) Foreign Application Priority Data
Apr. 11, 2023 (BE) BE2023/5272

(51) Int. Cl. A21D 8/04; C12N 9/18; C12Q 1/44

(52) U.S. Cl. CPC A21D 8/042; C12N 9/18; C12Q 1/44; C12Y 301/01026

(57) ABSTRACT

The present invention provides a polypeptide with galactolipase activity, characterized in that the ratio galactolipase activity versus phospholipase activity is 100.0 or higher and/or the ratio galactolipase activity versus lipase activity is 150.0 or higher. Also provided herein is an isolated nucleic acid encoding such polypeptide with galactolipase activity, an expression vector comprising such nucleic acid and a host cell comprising such nucleic acid or expression vector. Further provided herein is the use of said polypeptide with galactolipase activity as a food additive or in preparing a food product, and a method for preparing a food product using said polypeptide with galactolipase activity. Further provided herein is a dough product, batter product or bakery product obtained by a use or a method as described herein.

### CROSS REFERENCE TO RELATED APPLICATIONS

This application is a national phase entry under 35 U.S.C. § 371 of International Patent Application PCT/EP2024/059770, filed Apr. 11, 2024, designating the United States of America and published in English as International Patent Publication WO2024/213600 on Oct. 17, 2024, which claims the benefit under Article 8 of the Patent Cooperation Treaty to Belgian Patent Application Serial No. BE2023/5272, filed Apr. 11, 2023, the entireties of which are hereby incorporated by reference.

### INCORPORATION BY REFERENCE

The ST.26 XML Sequence listing named “PURA ̊537 ̊PCT.xml”, created on Apr. 10, 2024, and having a size of 12,288 bytes, is hereby incorporated herein by this reference in its entirety.

### FIELD OF THE INVENTION

The present invention relates to new polypeptides with galactolipase activity, especially to polypeptides with galactolipase activity with improved properties for use in dough, in which they may substitute emulsifiers, but not limited thereto.

### BACKGROUND OF THE INVENTION

In the past few decades, the demand for longer shelf ̊life and consistent quality in baked goods leads to the use of a wide range of additives in the baking industry. These additives, also referred to as bread improvers, include for example emulsifiers, enzymes, soy flour, oxidants and reductants, and are essential for improving dough machinability, reducing resting time and improving the baked goods' shelf life, volume, crust colour, crumb whiteness, aroma and flavour.

One important aspect of the baking process is controlling the proofing and the handling of doughs. Indeed, when doughs are over ̊proofed or when doughs incur shocks during handling, the quality of the baked products can be seriously affected. Therefore the skilled person is constantly searching for ways to improve dough “tolerance”.

Dough tolerance may therefore be defined as its ability to survive excessive fermentation times during proofing, mechanical shocks and different kind of process stresses like freezing, etc. Improved tolerance allows the baker to obtain bakery products with a better and more uniform quality. Tolerance is usually linked to the volume of the baked product. Volume is, inter alia, linked to the presence of a strongly developed gluten network. Emulsifiers are known to help contributing strength to this network.

Diacetyl tartaric esters of mono ̊glycerides (DATEM) are emulsifiers that have been widely used as bread improvers. DATEM improve bread volume and texture as well as dough stability.

In situ generation of emulsifying molecules may be performed by other means. For example, lipases have been used over the past three decades to improve some properties of baked goods like dough stability. They hydrolyse triglyceride esters and produce mono ̊ or di ̊glycerides, glycerol and free fatty acids. Lipases strengthen dough stability and increase bread volume, texture and shelf life. In the 1990s, the first generation of lipases (e.g. lipase of Thermomyces lanuginosus) typically hydrolyse the ester bond between glycerides and fatty acids in positions 1 and 3 in triglycerides, producing free fatty acids and mono ̊glycerides, and producing more polar lipids in the dough. While strengthening the gluten network, their overdosing stiffens the dough and decreases the loaf volume. The second generation lipases (e.g. Fusarium oxysporum lipase) works on both polar and non ̊polar lipids of the wheat flour, producing components with an even increased polarity, such as lysolecithin and digalactosyl monoacyl glycerol (DGMG). Lipases of the third generation (e.g. engineered Thermomyces lanuginosus lipase) have a lower affinity for short chain fatty acids and increase the expansion of the gluten network and the wall thickness of the crumb cells and decrease the crumb cell density, thereby improving the properties of baked products.

Although lipases and/or phospholipases have already been described to improve dough tolerance, the outcome of their use remains highly unpredictable, due to their different specificities, their different hydrolysis products, their potential synergies or the process conditions or substrates. This is particularly true for baked products made from doughs based on wholemeal flour where no satisfactory enzymatic solutions have been described so far. Nowadays the consumers prefer “healthier” baked products with a high(er) nutritional value. Compared to white flour, the use of wholemeal flour in baked products allows improving their nutritional profile, through the availability of more fibres, proteins and vitamins.

Accordingly, there is still a need for new compositions and methods to further improve the properties of (wholemeal) baked products, such as the volume, through improving dough or batter tolerance.

### SUMMARY OF THE INVENTION

The present inventors have surprisingly found that a polypeptide with galactolipase activity and with low or almost completely absent lipase activity and/or low or almost completely absent phospholipase activity has a positive effect on the properties of a dough or batter (e.g. dough tolerance) and/or on the properties of a baked product obtained from such dough or batter (e.g. volume after mechanical shock), in particular when the dough or batter comprises wholemeal flour.

Accordingly, a first aspect provides a polypeptide with galactolipase activity, characterized in that the ratio galactolipase activity versus phospholipase activity is 100.0 or higher and/or the ratio galactolipase activity versus lipase activity is 150.0 or higher.

In particular embodiments, the ratio galactolipase activity versus phospholipase activity is 1000.0 or higher.

In particular embodiments, the ratio galactolipase activity versus lipase activity is 300.0 or higher, preferably 500.0 or higher, and more preferably 1000.0 or higher.

In particular embodiments, the polypeptide with galactolipase activity is selected from:
(a) a polypeptide comprising a sequence having at least 80.0% sequence identity to the amino acid sequence of SEQ ID NO: 2 or SEQ ID NO: 4, preferably SEQ ID NO: 4;
(b) a polypeptide comprising a sequence encoded by a polynucleotide comprising a sequence having at least 80.0% sequence identity to the nucleotide sequence of SEQ ID NO:1 or SEQ ID NO: 3, preferably SEQ ID NO: 3;
(c) a fragment of the polypeptide of (a) or (b), having galactolipase activity, or;
(d) a polypeptide having at least 90.0% structural similarity with the structure of the polypeptide comprising the amino acid sequence of SEQ ID NO: 2 or SEQ ID NO: 4, preferably SEQ ID NO: 4.

In particular embodiments, the three dimensional protein coordinates of the atoms of its amino acid sequence are defined by the set of atomic structural coordinates displayed in FIG. 1 or have a root mean square deviation from the set of atomic structural coordinates displayed in FIG. 1 of not more than 1.8 Å.

In particular embodiments, said polypeptide is a Clostridium galactolipase.

A further aspect provides the use of a polypeptide with galactolipase activity as taught herein as a food additive or in preparing a food product.

In particular embodiments, the food product is (a) a dough or a dough product, (b) a batter or a batter product; or (c) a bakery or patisserie product.

In particular embodiments, the food additive is a bread improver.

In particular embodiments, said food product further comprises wholemeal flour.

In particular embodiments, the use as taught herein is to increase the tolerance of a dough or a batter.

A further aspect provides a method for preparing a food product comprising the step of admixing a polypeptide with galactolipase activity as taught herein with a food ingredient.

In particular embodiments, the food product is a bakery or patisserie product and the polypeptide with galactolipase activity as taught herein is added to a dough or batter.

In particular embodiments, said food product further comprises wholemeal flour.

A further aspect provides a food product, dough product, batter product or bakery product obtained by a use or method as taught herein.

A further aspect provides an isolated nucleic acid encoding a polypeptide with galactolipase activity as taught herein, preferably comprising a sequence having at least 80.0% sequence identity to SEQ ID NO:1 or SEQ ID NO: 3, preferably SEQ ID NO: 3.

A further aspect provides an expression vector comprising the nucleic acid as taught herein.

A further aspect provides a host cell comprising the nucleic acid as taught herein or an expression vector as taught herein, preferably wherein the host cell is a bacterial, fungal or yeast cell.

A further aspect provides a method for identifying a polypeptide with galactolipase activity for improving dough tolerance, the method comprising the step of determining whether a polypeptide has at least 90.0% structural similarity with the structure of the polypeptide comprising the amino acid sequence of SEQ ID NO: 2 or SEQ ID NO: 4, preferably SEQ ID NO: 4.

A further aspect provides a method for identifying a polypeptide with galactolipase activity for improving dough tolerance, the method comprising the step of determining whether the three ̊dimensional protein coordinates of the atoms of the amino acid sequence of a polypeptide have a root mean square deviation from the set of atomic structural coordinates displayed in FIG. 1 of not more than 1.8 Å.

### BRIEF DESCRIPTION OF THE FIGURES

FIGS. 1A-1AX provides a table encompassing the atomic structural coordinates of amino acid residues 1 ̊349 of SEQ ID NO: 4, as predicted by AlphaFold.

Column 1: record type (ATOM), column 2: atom serial number, column 3: atom name, column 4: residue name, column 5: chain identifier, column 6: residue sequence number, column 7: atomic coordinate X in Angstroms, column 8: atomic coordinate Y in Angstroms, column 9: atomic coordinate Z in Angstrom, column 10: occupancy, column 11: temperature factor, column 12: element symbol

FIGS. 2A-2D provides the sequences as depicted in SEQ ID NO: 1, SEQ ID NO: 2, SEQ ID NO: 3, SEQ ID NO: 4, SEQ ID NO: 5, SEQ ID NO:6, and SEQ ID NO: 7.

### DESCRIPTION

Before the present method and devices used in the invention are described, it is to be understood that this invention is not limited to particular methods, components, or devices described, as such methods, components, and devices may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any method and material similar or equivalent to those described herein may be used in practice or testing of the present invention, the preferred methods and materials are now described.

In this specification and the appended claims, the singular forms “a”, “an”, “the” include both the singular and the plural, unless the context clearly indicates otherwise.

The terms “comprising”, “comprises” and “comprised of” as used herein are synonymous with “including”, “includes” or “containing”, “contains”, and are inclusive or open ̊ended and do not exclude additional, non ̊recited members, elements or method steps. Where this description refers to a product or process which “comprises” specific features, parts or steps, this refers to the possibility that other features, parts or steps may also be present, but may also refer to embodiments which only contain the listed features, parts or steps. The terms “comprising”, “comprises” and “comprised of” also include the term “consisting of”.

The enumeration of numeric values by means of ranges of figures comprises all values and fractions in these ranges, as well as the cited end points.

The terms “about” and “approximately” as used when referring to a measurable value, such as a parameter, an amount, a time period, and the like, is intended to include variations of +/ ̊10% or less, preferably +/ ̊5% or less, more preferably +/ ̊1% or less, and still more preferably +/ ̊0.1% or less, of and from the specified value, in so far as the variations apply to the invention disclosed herein. It should be understood that the value to which the term “about” or “approximately” refers per se has also been disclosed.

The inventors have surprisingly found that a polypeptide with galactolipase activity and a low or almost completely absent lipase and/or phospholipase activity has a positive effect on the dough properties or on the baked product properties obtained therefrom. It was further surprisingly found that the use of such a polypeptide with galactolipase activity increases the tolerance of the dough, in particular when the dough comprises wholemeal flour.

It is therefore a first aspect of the present invention to provide a polypeptide with galactolipase activity (or galactolipase), characterized in that the ratio galactolipase activity versus phospholipase activity (i.e. ratio galactolipase activity over phospholipase activity) of the polypeptide (or galactolipase) is 100.0 or higher and/or the ratio galactolipase activity versus lipase activity of the polypeptide (or galactolipase) is 150.0 or higher.

In other words, provided herein is a polypeptide having galactolipase activity (or galactolipase) characterized in that the ratio galactolipase activity/phospholipase activity (ratio between galactolipase activity and phospholipase activity) is 100.0 or higher and/or the ratio galactolipase activity/lipase activity is 150.0 or higher.

In some embodiments the ratio galactolipase activity versus phospholipase activity is 300.0 or higher, preferably 500.0 or higher, more preferably 1000.0 or higher. In some embodiments the ratio galactolipase activity versus lipase activity is 300.0 or higher, preferably 500.0 or higher and more preferably 1000.0 or higher. In some embodiments the ratio galactolipase activity versus phospholipase activity is 100.0 or higher, preferably 300.0 or higher, more preferably 500.0 or higher, even more preferably 1000.0 or higher, and the ratio galactolipase activity versus lipase activity is 150.0 or higher, preferably 300.0 or higher, more preferably 500.0 or higher, even more preferably 1000.0 or higher.

Preferably the ratios are obtained when determining the enzymatic activity as described herein. Furthermore, in particular embodiments, the galactolipase activity is determined at 30° C. and/or the phospholipase activity is determined at 30° C.

In the context of the present invention the term “polypeptide having galactolipase activity” or “polypeptide with galactolipase activity” or “galactolipase” (EC 3.1.1.26) refers to enzymes that catalyse, as main enzymatic activity, the cleavage of acyl ester bonds of galactolipids, such as monogalactosyldiacylglycerols (MGDG) or digalactosyldiacylglycerols (DGDG), forming free fatty acids (FFAs), digalactosylglycerol, and/or lysogalactolipids (LysoGL). Galactolipase activity may be assayed using different methods such as pHstat titration with dioctanoyl galactolipid ̊bile salt mixed micelles, barostat with dilauroyl galactolipid monomolecular films spread at the air ̊water interface, UV absorption using a novel monogalactosyldiacylglycerol (MGDG) substrate containing α ̊eleostearic acid as chromophore and coated on microtiter plates. Preferably the galactolipase activity may be measured using digalactosyldiacylglycerol (DGDG) as substrate. The release of free fatty acid due to hydrolysis of digalactosyldiacylglycerol by galactolipase is measured by spectrophotometry at 550 nm. One galactolipase activity (SDGU) is defined as the amount of enzyme needed to release 1 nmol of free fatty acid per minute at 30° C. and pH 7.5. For example, the galactolipase activity may be determined as exemplified in Example 1.

The term further also includes polypeptide variants or mutants which carry amino acid sequence variations vis ̊à ̊vis a corresponding native polypeptide, such as, e.g., amino acid deletions, additions and/or substitutions, as described elsewhere herein. The term contemplates both full ̊length polypeptides and polypeptide parts or fragments, e.g., naturally ̊occurring polypeptide parts that ensue from processing of such full ̊length polypeptides, as described elsewhere herein.

In the context of the present invention the term “lipase” (EC 3.1.1.3) refers to enzymes having, as main enzymatic activity, triacylglycerol lipase activity. Lipase activity may be assayed using different methods such as fluorometric, colorimetric or titrimetric methods. Preferably the lipase activity may be measured using p ̊nitrophenyl palmitate (pNPP) as substrate. The release of yellow p ̊nitrophenol due to hydrolysis of p ̊nitrophenyl palmitate by lipase is measured by spectrophotometry at 414 nm. One lipase milliunit (LmU) is defined as the amount of enzyme needed to release one nanomole (nmole) per minute of p ̊nitrophenol from p ̊nitrophenyl palmitate at 45° C. and pH 7.5. For example, the lipase activity may be determined as exemplified in Example 1.

In the context of the present invention the term “phospholipase” refers to enzymes having, as main enzymatic activity, hydrolytic activity towards one or both carboxylic ester bonds in phospholipids, such as for example phospholipase A1 or phospholipase A2 (respectively EC 3.1.1.32 or 3.1.1.4). Phospholipase activity may be assayed using different methods such as Phospholipase Activity Assay Kit (Colorimetric, Abcam), Secretory Phospholipase Activity Assay Kit (Fluorometric, Abcam), EnzChek® Phospholipase A1/A2 Assay Kit (Life Technologies). Preferably the phospholipase activity may be measured using 1,2 ̊Dioleoyl ̊sn ̊glycero ̊3 ̊phospho ̊rac ̊1 ̊glycerol (DOPG) as substrate. The release of free fatty acid due to hydrolysis of 1,2 ̊Dioleoyl ̊sn ̊glycero ̊3 ̊phospho ̊rac ̊1 ̊glycerol by phospholipase is measured by spectrophotometry at 550 nm. One phospholipase activity unit (NefU) is defined as the amount of enzyme needed to release 1 nmol of free fatty acid per minute at 30° C. and pH 7.5. For example, the phospholipase activity may be determined as exemplified in Example 1.

In particular embodiments, the polypeptide with galactolipase activity as taught therein is selected from:
(a) a variant of the polypeptide with galactolipase activity as taught herein, such as a polypeptide comprising, consisting essentially of or consisting of a sequence having at least 80.0%, preferably at least 95.0%, sequence identity to the amino acid sequence as defined in (i.e. the amino acid sequence of) SEQ ID NO: 2, SEQ ID NO: 4 or SEQ ID NO: 6, preferably SEQ ID NO: 2 or SEQ ID NO: 4, more preferably SEQ ID NO: 4;
(b) a polypeptide comprising, consisting essentially of or consisting of a sequence encoded by a polynucleotide comprising, consisting essentially of or consisting of a sequence having at least 80.0%, preferably at least 95.0%, sequence identity to the nucleotide sequence as defined in SEQ ID NO:1, SEQ ID NO:3, or SEQ ID NO: 5, preferably SEQ ID NO: 1 or SEQ ID NO: 3, more preferably SEQ ID NO: 3;
(c) a fragment of the polypeptide of (a) or (b) having galactolipase activity, and/or
(d) a polypeptide having at least 90.0%, preferably at least 95.0%, structural similarity with the structure of the polypeptide comprising, consisting essentially of or consisting of the amino acid sequence as defined in SEQ ID NO: 2, SEQ ID NO: 4 or SEQ ID NO: 6, preferably SEQ ID NO: 2 or SEQ ID NO: 4, more preferably SEQ ID NO: 4.

The term “variant” of a protein, polypeptide, peptide or nucleic acid generally refers to proteins, polypeptides or peptides the amino acid sequence of which, or nucleic acids the nucleotide sequence of which, is substantially identical (i.e., largely but not wholly identical) to the sequence of the protein, polypeptide, peptide, or nucleic acid, e.g., at least about 80.0% identical or at least about 85.0% identical, e.g., preferably at least about 90.0% identical, e.g., at least 91.0% identical, 92.0% identical, more preferably at least about 93.0% identical, e.g., at least 94.0% identical, even more preferably at least about 95.0% identical, e.g., at least 96.0% identical, yet more preferably at least about 97.0% identical, e.g., at least 98.0% identical, and most preferably at least 99.0% identical to the sequence of the recited protein, polypeptide, peptide, or nucleic acid. Preferably, a variant may display such degrees of identity to a recited protein, polypeptide, peptide or nucleic acid when the whole sequence of the recited protein, polypeptide, peptide or nucleic acid is queried in the sequence alignment (i.e., overall sequence identity).

In the context of the present invention the term “sequence identity” corresponds to the relatedness between two amino acid or nucleotide sequences.

For purposes of the present invention, sequence identity may be determined using suitable algorithms for performing sequence alignments and determination of sequence identity as know per se. Exemplary but non ̊limiting algorithms include those based on the Basic Local Alignment Search Tool (BLAST) originally described by Altschul et al. 1990 (J Mol Biol 215: 403 ̊10), such as the “Blast 2 sequences” algorithm described by Tatusova and Madden 1999 (FEMS Microbiol Lett 174: 247 ̊250), for example using the published default settings or other suitable settings (such as, e.g., for the BLASTN algorithm: cost to open a gap=5, cost to extend a gap=2, penalty for a mismatch= ̊2, reward for a match=1, gap x_dropoff=50, expectation value=10.0, word size=28; or for the BLASTP algorithm: matrix=Blosum62 (Henikoff et al., 1992, Proc Natl Acad Sci 89:10915 ̊10919), cost to open a gap=11, cost to extend a gap=1, expectation value=10.0, word size=3).

An example procedure to determine the percent identity between a particular amino acid sequence and the amino acid sequence of a query polypeptide will entail aligning the two amino acid sequences using the BLASTP algorithm (with the BLAST ̊2 ̊Sequences options), available as a web application or as a standalone executable programme (BLAST version 2.13.0) at the NCBI web site (https://ftp.ncbi.nlm.nih.gov/blast/executables/blast+/LATEST/), using suitable algorithm parameters. If the two compared sequences share homology, then the output will present those regions of homology as aligned sequences. If the two compared sequences do not share homology, then the output will not present aligned sequences. Once aligned, the number of matches will be determined by counting the number of positions where an identical amino acid residue is presented in both sequences. The percent identity is determined by dividing the number of matches by the length of the query polypeptide, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by Bl2seq already conveniently includes the percentage of identities.

Sequence identity may also be determined using the Needleman ̊Wunsch algorithm (Needleman and Wunsch, 1970, J Mol Biol 48: 443 ̊453) as implemented in the Needle program of the EMBOSS package (EMBOSS: The European Molecular Biology Open Software Suite, Rice et al., 2000, Trends Genet. 16: 276 ̊277), preferably version 5.0.0 or later. The parameters used may be gap open penalty of 10, gap extension penalty of 0.5, and the EBLOSUM62 (EMBOSS version of BLOSUM62) substitution matrix for amino acid sequence comparison. The output of Needle labeled “longest identity” (obtained using the −nobrief option) may be used as the percent identity and may be calculated as follows: (Identical Residues×100)/(Length of Alignment−Total Number of Gaps in Alignment).

Sequence identity may also be determined using FASTA36 sequence comparison software, such as described in Pearson W.R., Finding protein and nucleotide similarities with FASTA, Curr Protoc Bioinformatics, 2016 Mar. 24; 53:3.9.1 ̊3.925.

A variant of a protein, polypeptide, or peptide may comprise one or more (e.g. several) amino acid additions, deletions, or substitutions relative to (i.e., compared with) the corresponding protein or polypeptide. A substitution means replacement of the amino acid occupying a position with a different amino acid; a deletion means removal of the amino acid occupying a position; and an insertion means adding an amino acid adjacent to and immediately following the amino acid occupying a position.

In some embodiments, the galactolipase (i.e. the polypeptide with galactolipase activity) as taught herein comprises, consists essentially of or consists of a sequence having at least 80.0%, at least 85.0% sequence identity, preferably at least 90.0%, more preferably at least 91.0%, at least 92.0%, at least 93.0%, at least 94.0%, at least 95.0%, at least 96.0%, at least 97.0%, at least 98.0%, at least 99.0% or 100.0% sequence identity with the sequence as defined by SEQ ID NO: 2 (comprising a signal peptide), SEQ ID NO: 4 (not comprising a signal peptide, or mature form of the galactolipase) or SEQ ID NO: 6 (not comprising a signal peptide, or mature form of the galactolipase), preferably SEQ ID NO: 2 or SEQ ID NO: 4, more preferably SEQ ID NO: 4.

SEQ ID NO: 6 corresponds to SEQ ID NO: 4 with 17 amino acid substitutions (and may be encoded by the nucleotide sequence as defined in SEQ ID NO:5). More particularly, the T residue at position 33 of SEQ ID NO: 4 is substituted by an I residue, the K residue at position 52 of SEQ ID NO: 4 is substituted by a R residue, the Y residue at position 120 of SEQ ID NO: 4 is substituted by a D residue, the D residue at position 132 of SEQ ID NO: 4 is substituted by a N residue, the S residue at position 181 of SEQ ID NO: 4 is substituted by a N residue, the A residue at position 184 of SEQ ID NO: 4 is substituted by a S residue, the G residue at position 194 of SEQ ID NO: 4 is substituted by a A residue, the K residue at position 239 of SEQ ID NO: 4 is substituted by a Q residue, the P residue at position 278 of SEQ ID NO: 4 is substituted by a L residue, the G residue at position 298 of SEQ ID NO: 4 is substituted by an A residue, the S residue at position 306 of SEQ ID NO: 4 is substituted by a T residue, the R residue at position 318 of SEQ ID NO: 4 is substituted by a I residue, the K residue at position 330 of SEQ ID NO: 4 is substituted by a A residue, the M residue at position 337 of SEQ ID NO: 4 is substituted by an I residue, the T residue at position 341 of SEQ ID NO: 4 is substituted by an S residue and the N residue at position 342 of SEQ ID NO: 4 is substituted by an M residue.

In particular embodiments, the polypeptide with galactolipase activity as taught herein comprises, consists essentially of or consists of a sequence that differs by at most 38, at most 37, at most 36, at most 35, at most 34, at most 33, at most 32, at most 31, at most 30, at most 25, at most 20, at most 15, at most 10, at most 9, at most 8, at most 7, at most 6, at most 5, at most 4, at most 3, at most 2, or at most 1 amino acid from the sequence as defined by SEQ ID NO: 2, SEQ ID NO: 4 or SEQ ID NO: 6, preferably SEQ ID NO: 2 or SEQ ID NO: 4, more preferably SEQ ID NO: 4. The term “differ by” may refer to amino acid substitutions, additions and/or deletions, preferably substitutions.

In particular embodiments, the variant of the polypeptide as taught herein is a functionally active variant of the polypeptide as taught herein.

The term “fragment” as used throughout this specification with reference to a peptide, polypeptide, or protein generally denotes a portion of the peptide, polypeptide, or protein, such as typically an N ̊ and/or C ̊terminally truncated form of the peptide, polypeptide, or protein. Preferably, a fragment may comprise at least about 30.0%, e.g., at least about 50.0% or at least about 70.0%, preferably at least about 80.0%, e.g., at least about 85.0%, more preferably at least about 90.0%, and yet more preferably at least about 95.0% or even about 99.0% of the amino acid sequence length of said peptide, polypeptide, or protein. For example, insofar not exceeding the length of the full ̊length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, of the corresponding full ̊length peptide, polypeptide, or protein.

The terms encompass fragments arising by any mechanism, in vivo and/or in vitro, such as, without limitation, by alternative transcription or translation, exo ̊ and/or endo ̊proteolysis, exo ̊ and/or endo ̊nucleolysis, or degradation of the peptide, polypeptide, protein or nucleic acid, such as, for example, by physical, chemical and/or enzymatic proteolysis or nucleolysis.

In particular embodiments, the fragment is a functionally active fragment of galactolipase (or polypeptide with galactolipase activity). By means of an example and not limitation, a functionally active fragment of the galactolipase as taught herein will at least partly retain one or more aspects of the functionality of the corresponding native or wild ̊type galactolipase as taught herein. For example, reference to the functionality of the galactolipase as taught herein may particularly denote its galactolipase activity, its very low or absent phospholipase activity and/or lipase activity, and/or the ability to increase the tolerance of a dough, in particular when the dough comprises wholemeal flour. The term “functionally active” as used herein denotes that the fragment and/or variant at least partly retains the intended functionality or biological activity of the respective or corresponding peptide, polypeptide or protein. Reference to the “functionality” or “activity” of a peptide, polypeptide or protein may generally encompass any one or more aspects of the functionality or biological activity of the peptide, polypeptide or protein, such as without limitation any one or more aspects of its biochemical activity, enzymatic activity, interaction activity, ligand activity, and/or structural activity, e.g., within a cell, tissue, organ or an organism.

Preferably, a functionally active fragment or variant may retain at least about 20.0%, e.g., at least about 25.0%, or at least 30.0%, or at least about 40.0%, or at least about 50.0%, e.g., at least 60.0%, more preferably at least about 70.0%, e.g., at least 80.0%, yet more preferably at least about 85.0%, still more preferably at least about 90.0%, and most preferably at least about 95.0% or even about 100.0% of the intended biological activity or functionality compared with the corresponding peptide, polypeptide or protein. In certain embodiments, a functionally active fragment or variant may even display higher biological activity or functionality compared with the corresponding peptide, polypeptide or protein, for example may display at least about 100.0%, or at least about 150.0%, or at least about 200.0%, or at least about 300.0%, or at least about 400.0%, or at least about 500.0% of the intended biological activity or functionality compared with the corresponding peptide, polypeptide or protein. By means of an example, where the activity of a given peptide, polypeptide or protein can be readily measured in an assay with a quantitative output, for example an enzymatic assay, a functionally active fragment or variant of the peptide, polypeptide or protein may produce a signal which is at least about 20.0%, or at least about 25.0%, or at least 30.0%, or at least about 40.0%, or at least about 50.0%, or at least 60.0%, more preferably at least about 70.0%, or at least 80.0%, or at least about 85.0%, or at least about 90.0%, or at least about 95.0%, or at least about 100.0%, or at least about 150.0%, or at least about 200.0%, or at least about 300.0%, or at least about 400.0%, or at least about 500.0% of the signal produced by the corresponding peptide, polypeptide or protein.

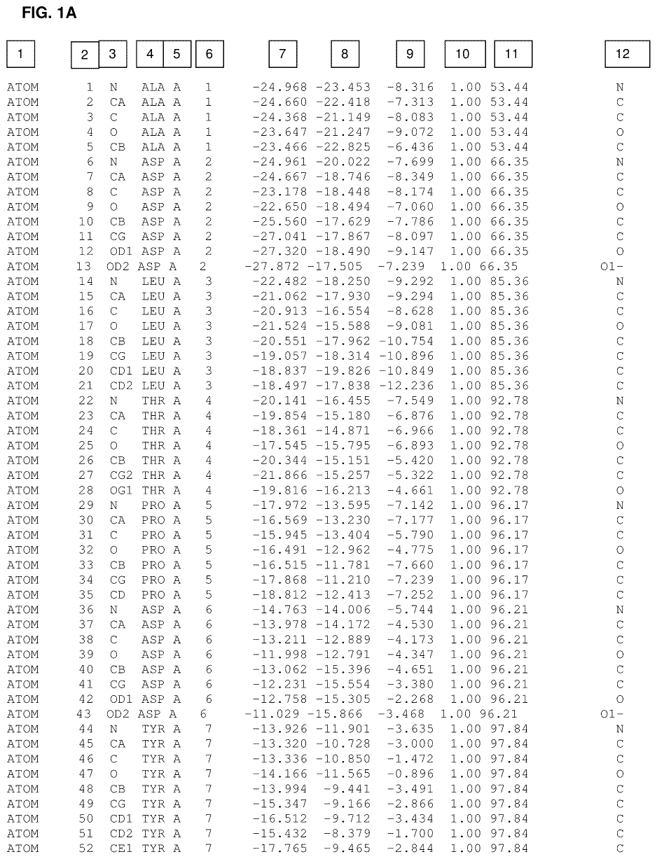

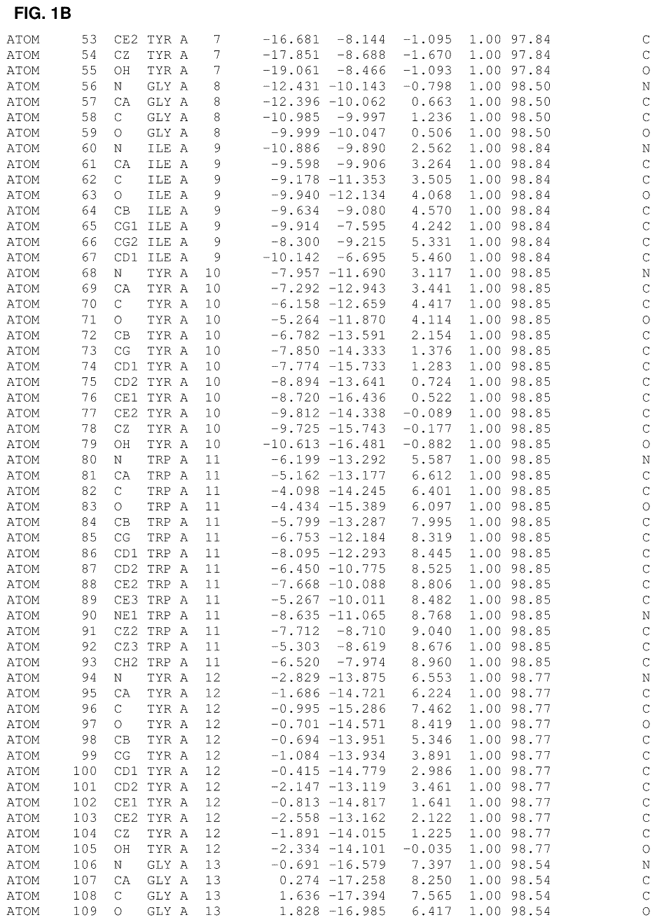

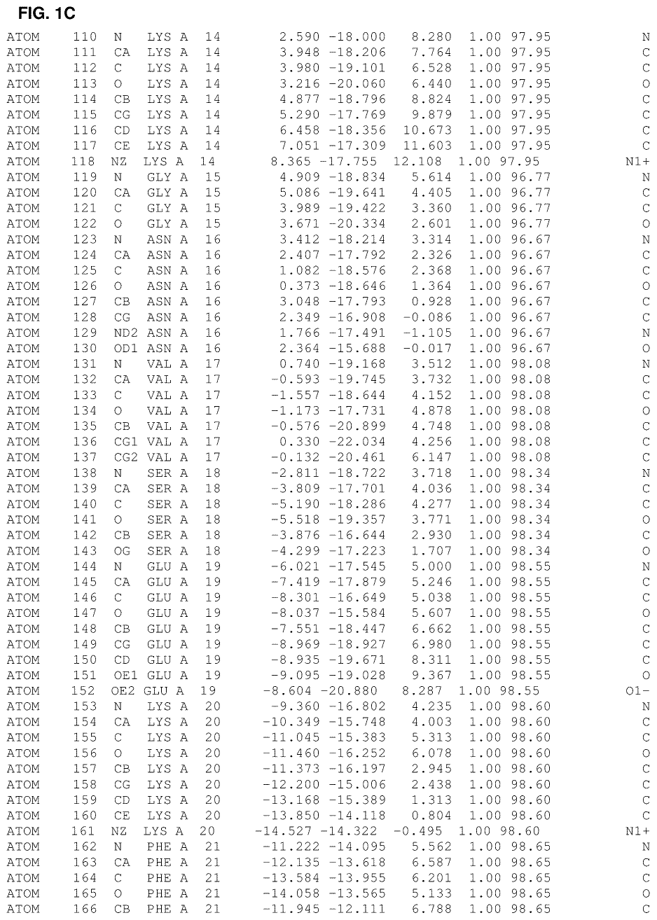

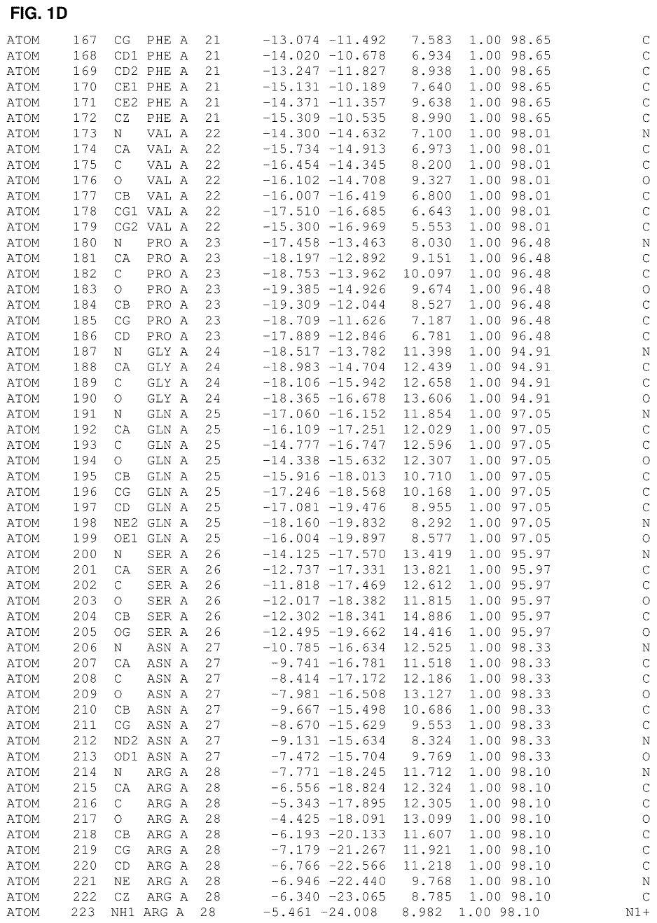

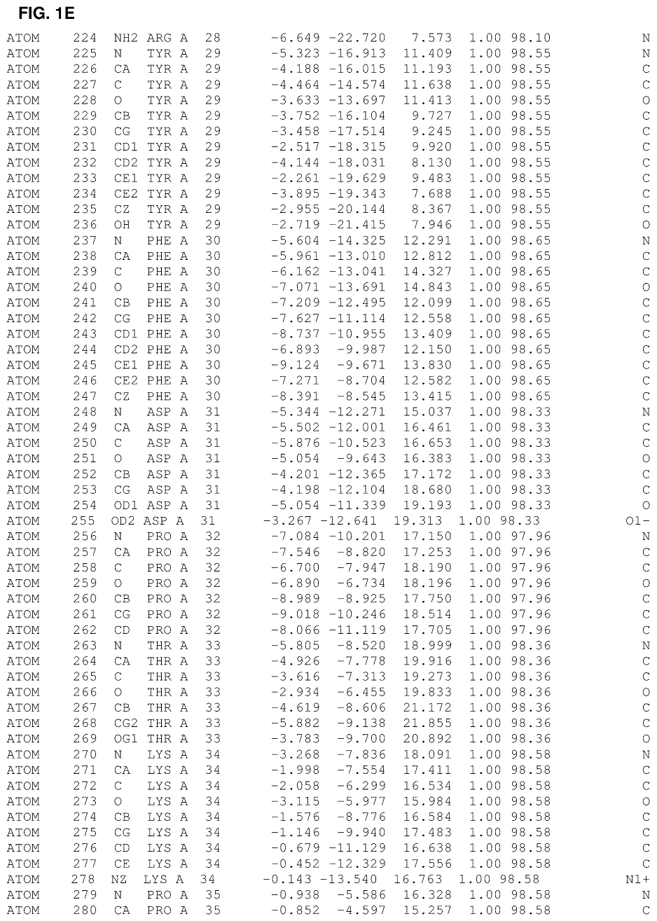

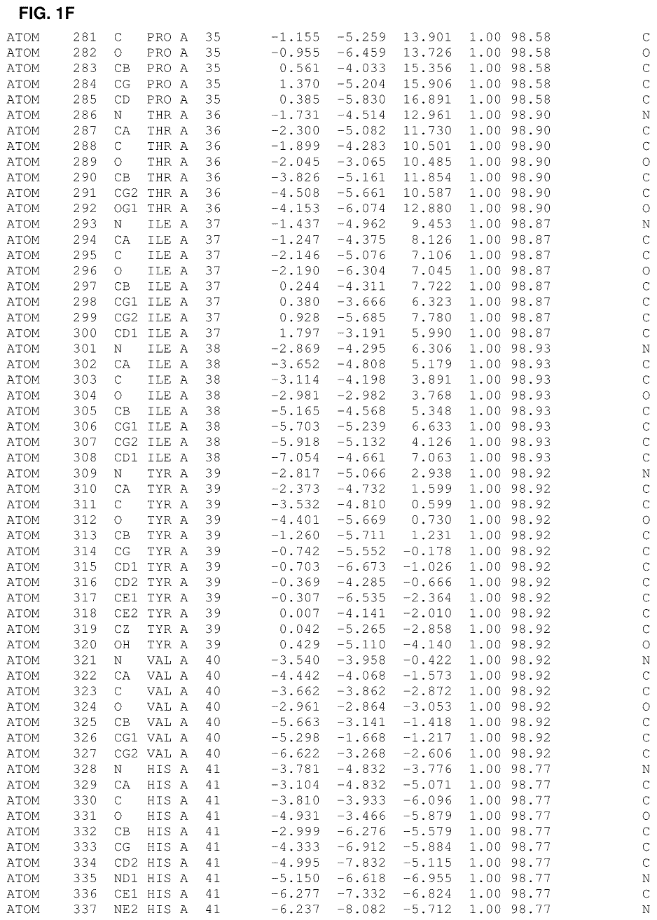

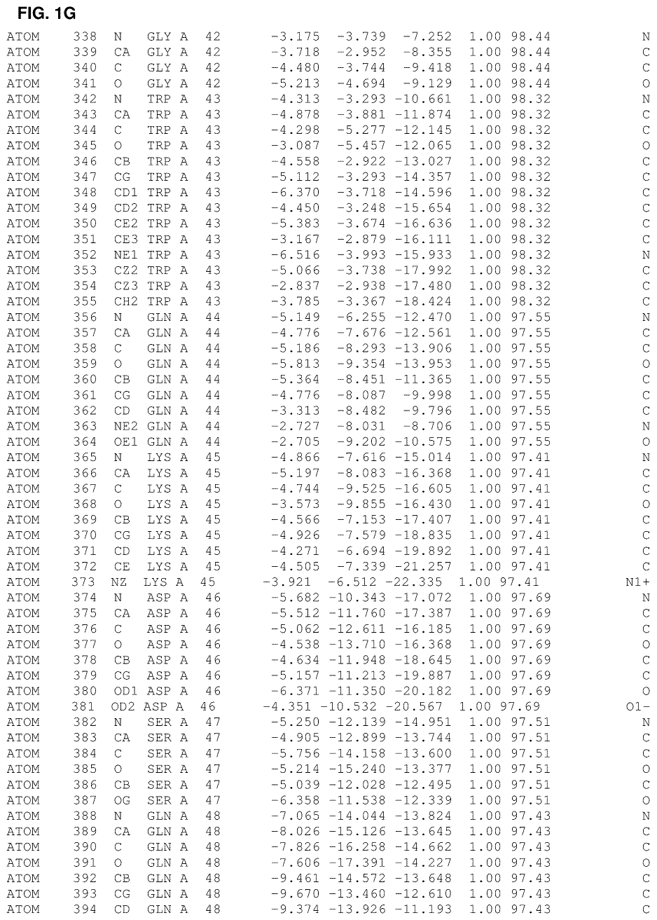

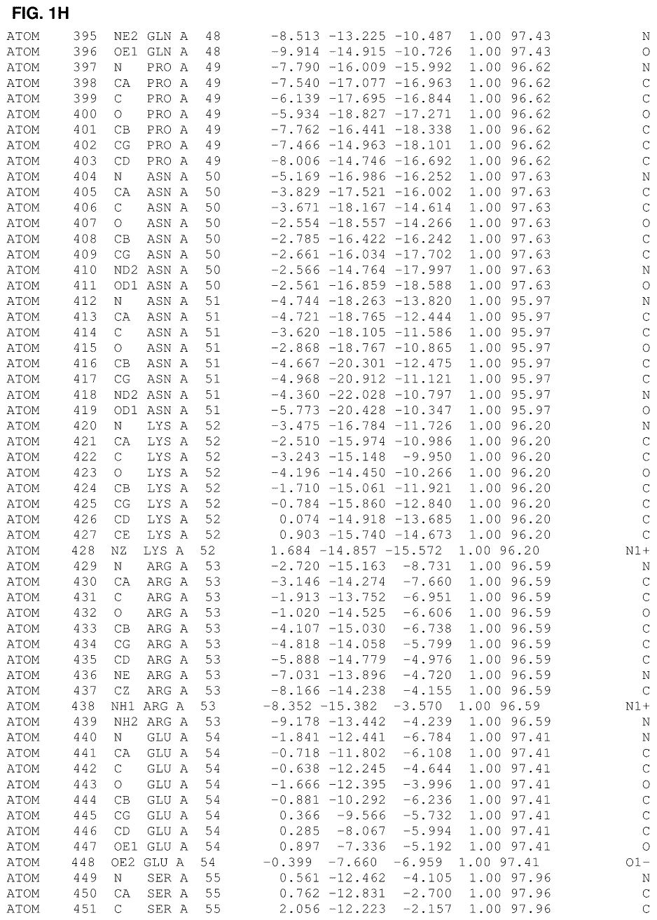

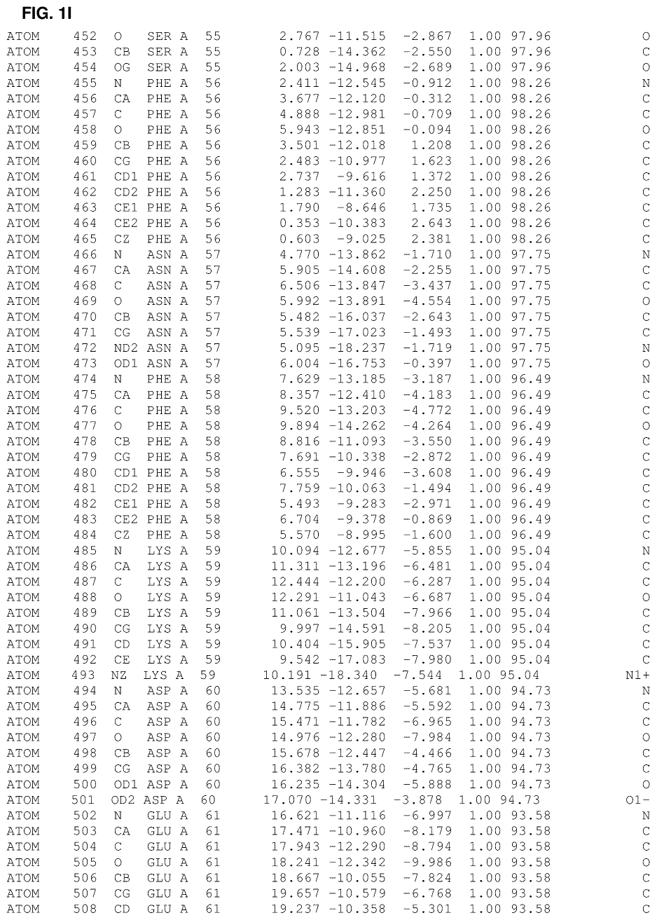

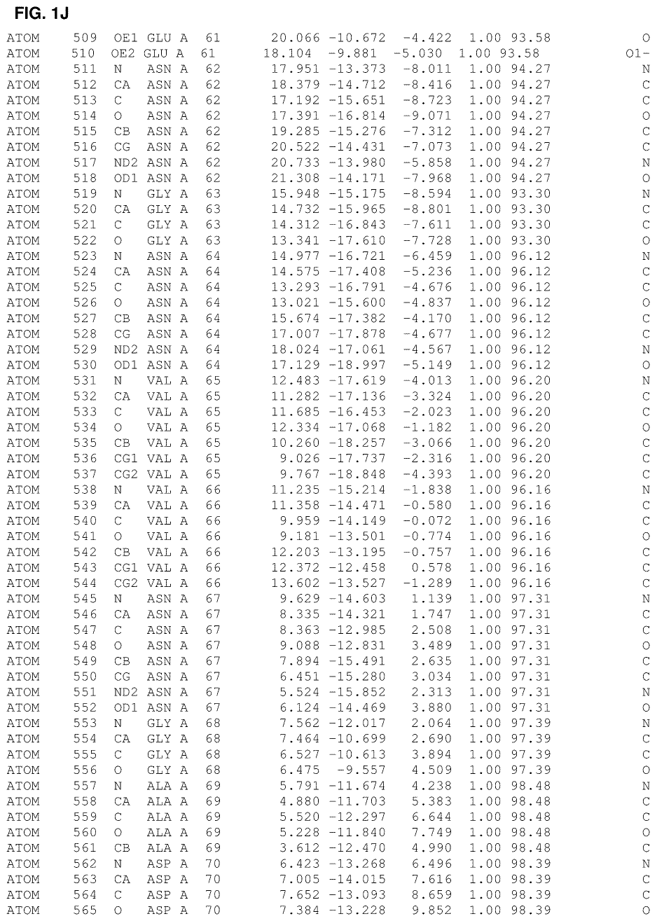

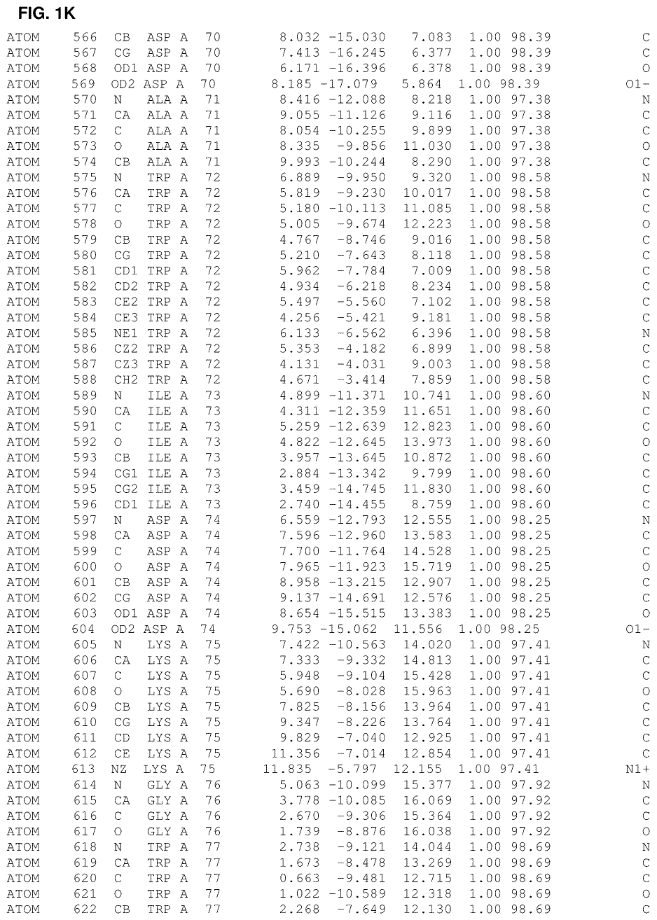

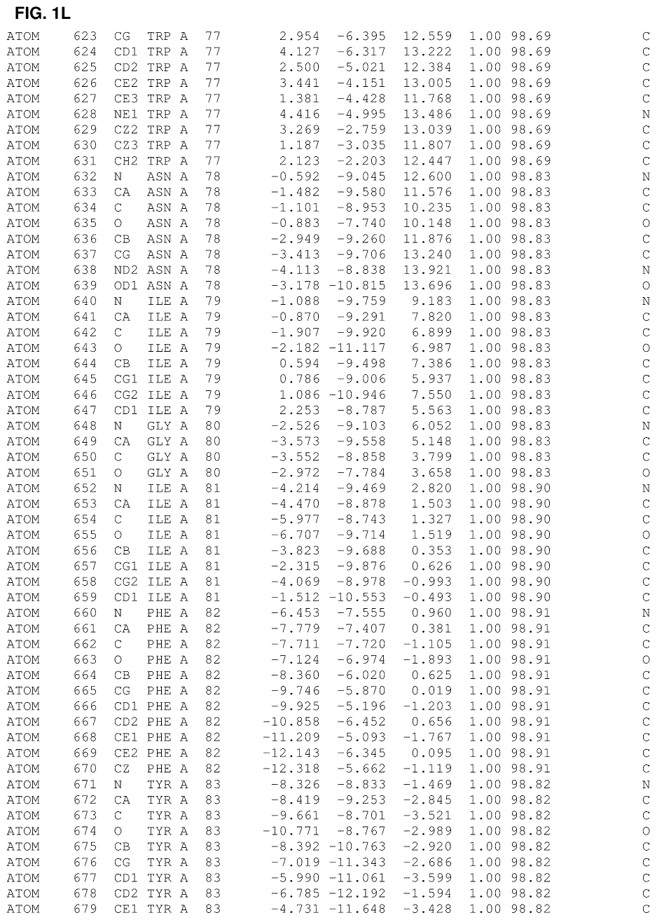

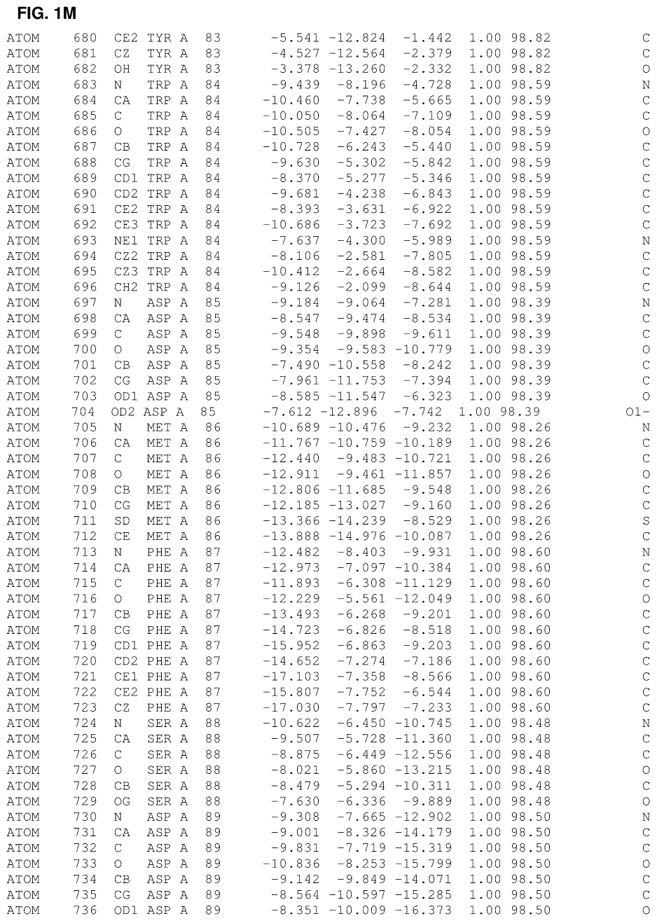

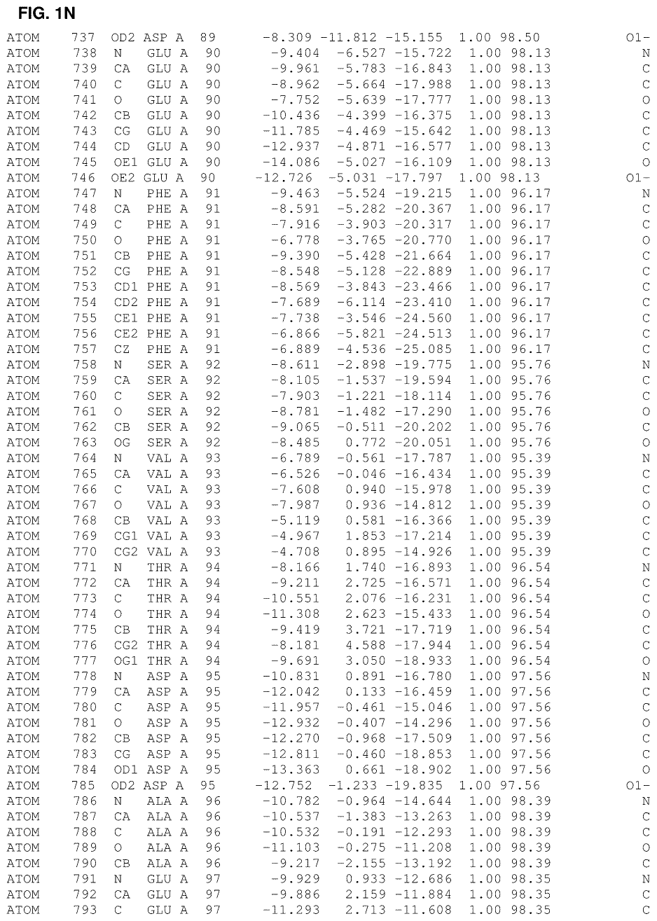

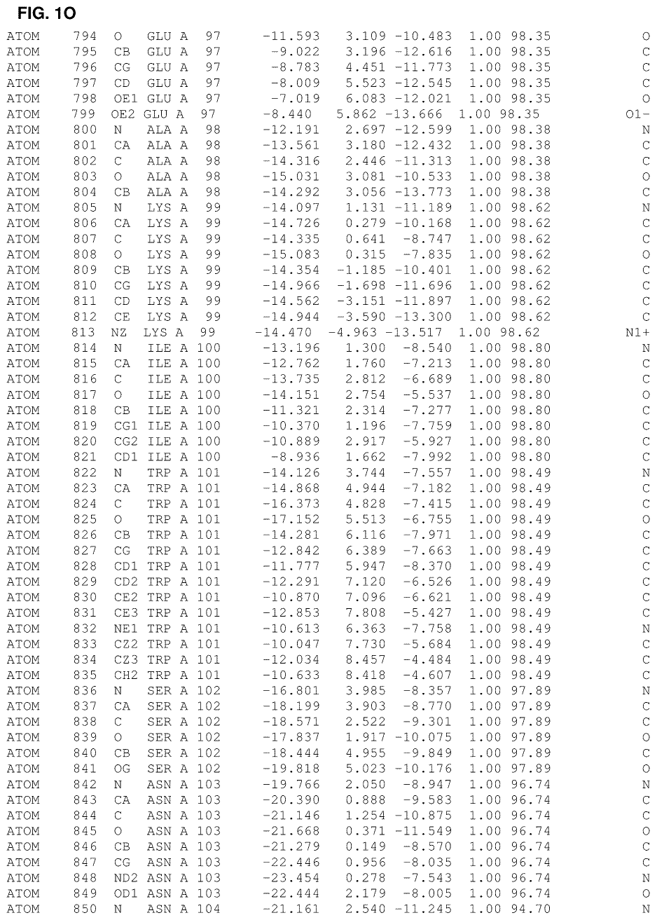

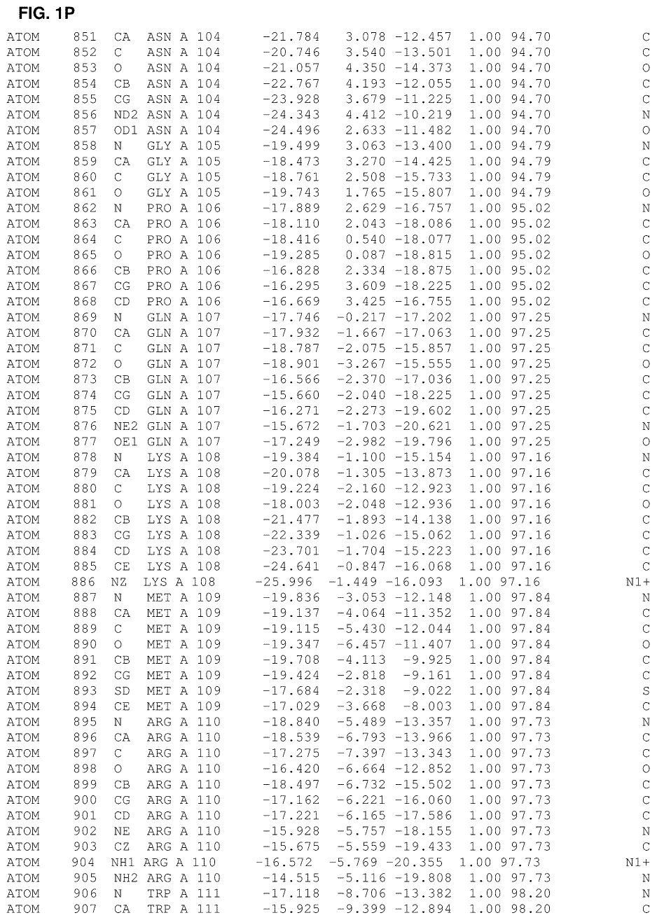

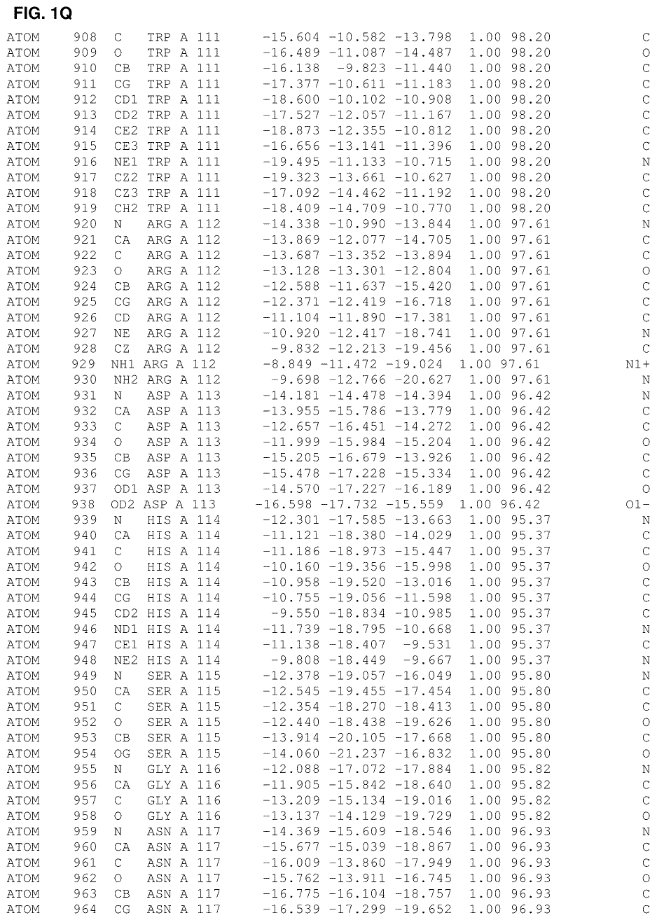

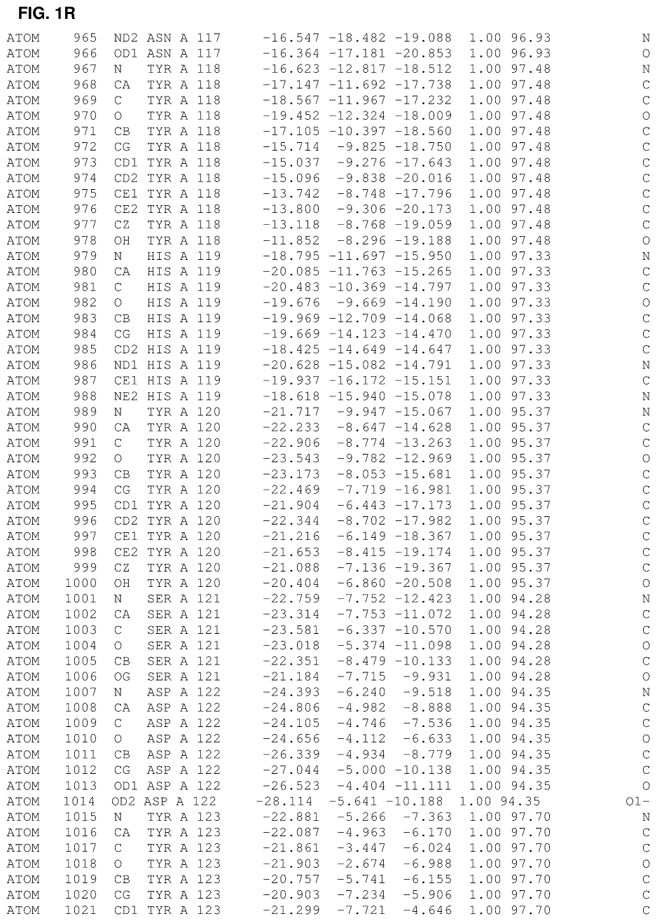

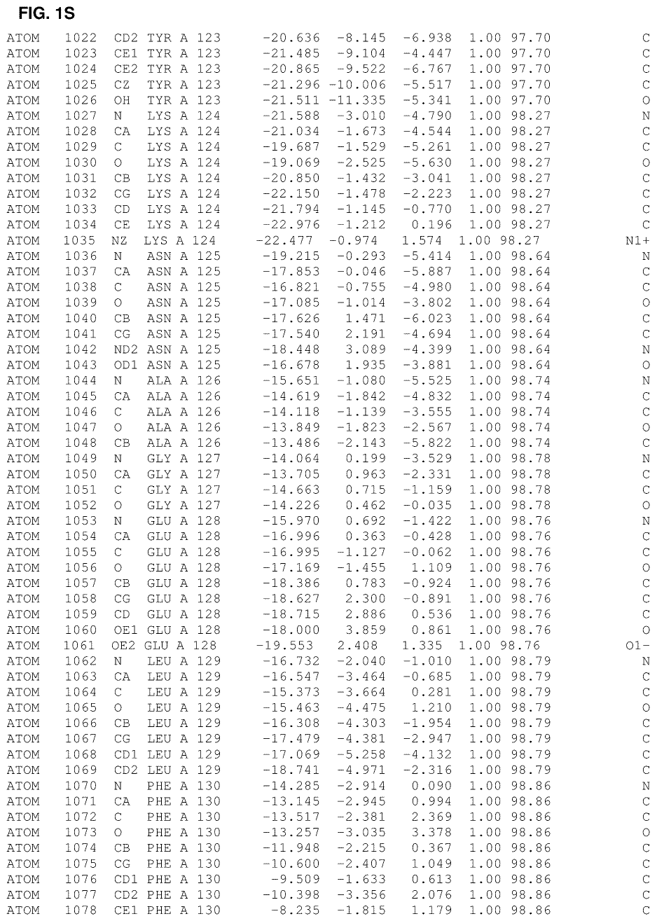

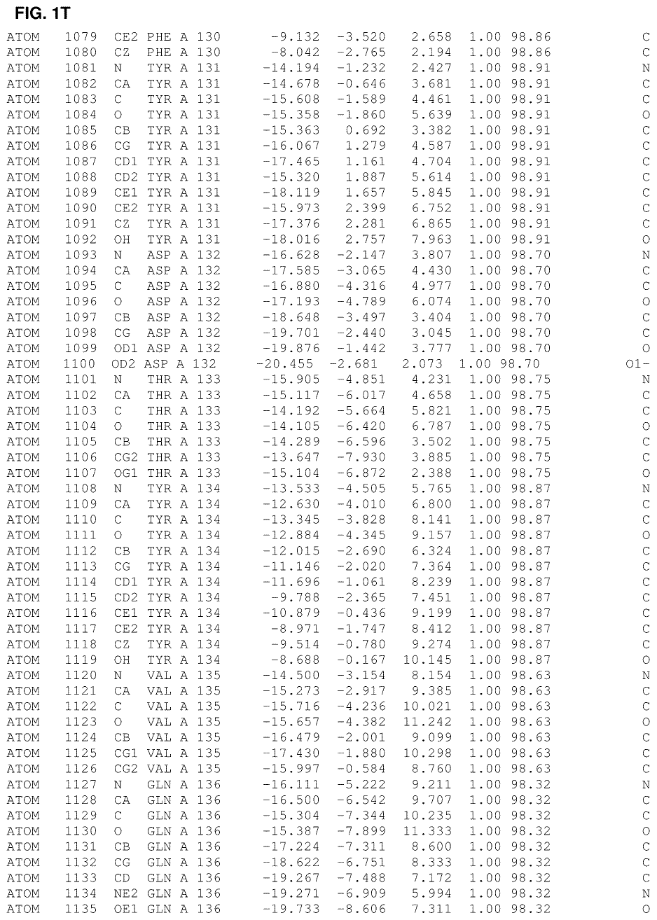

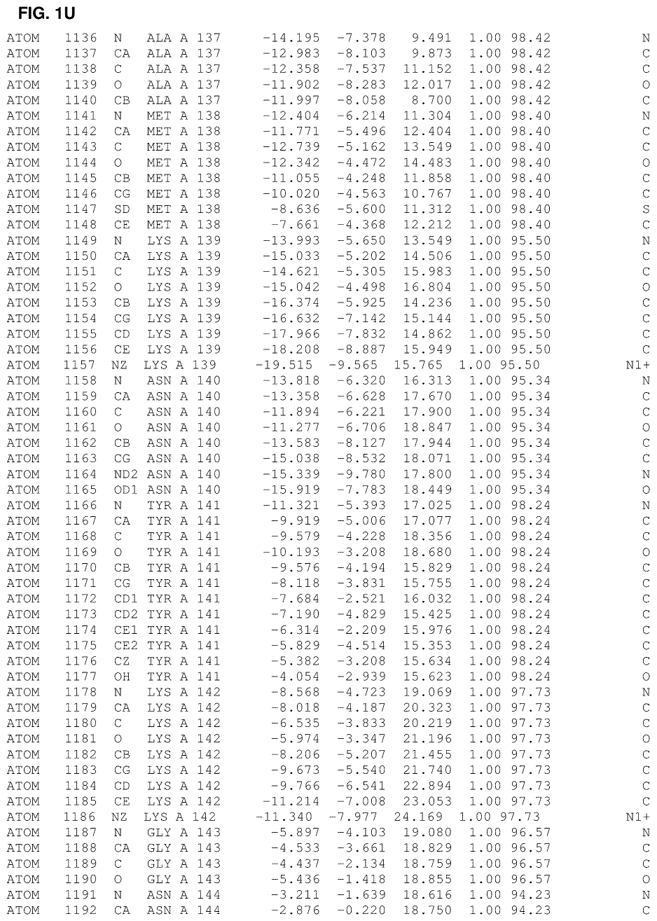

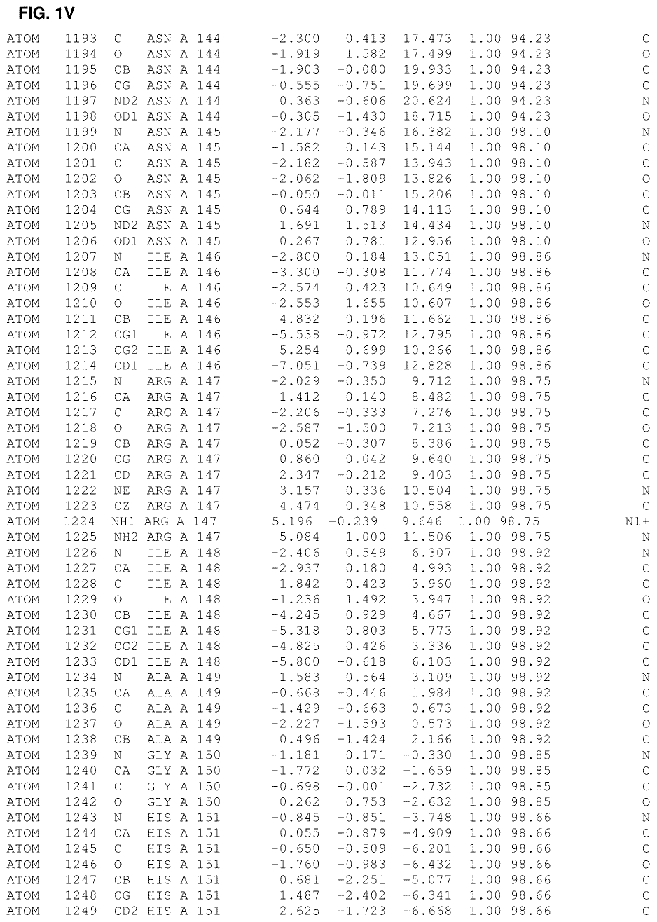

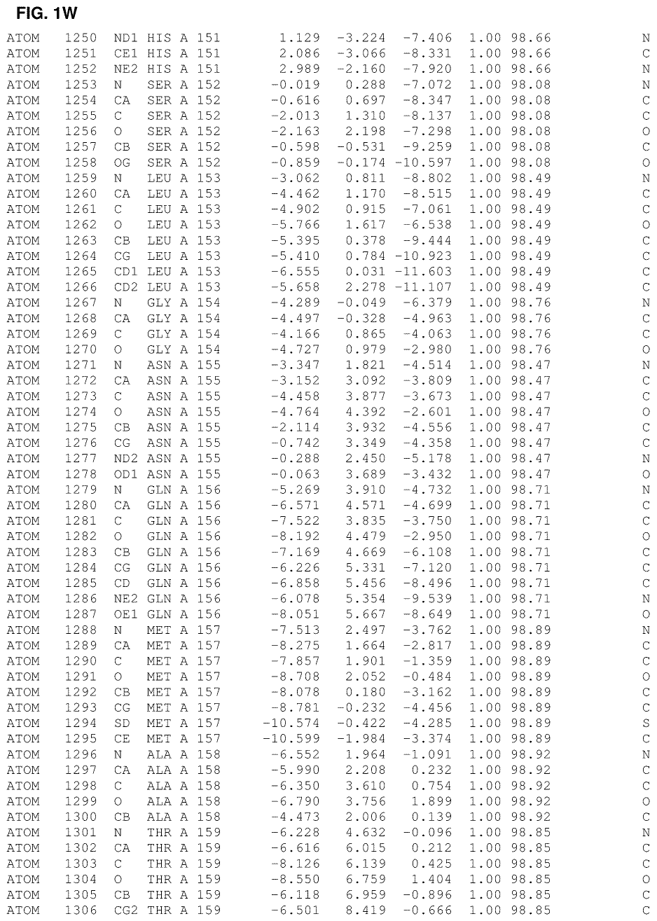

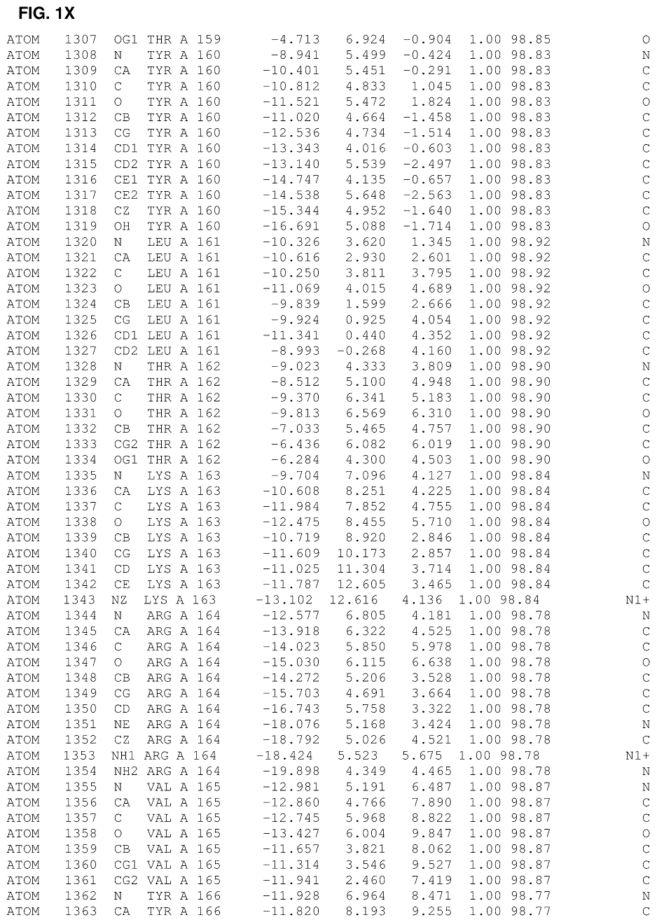

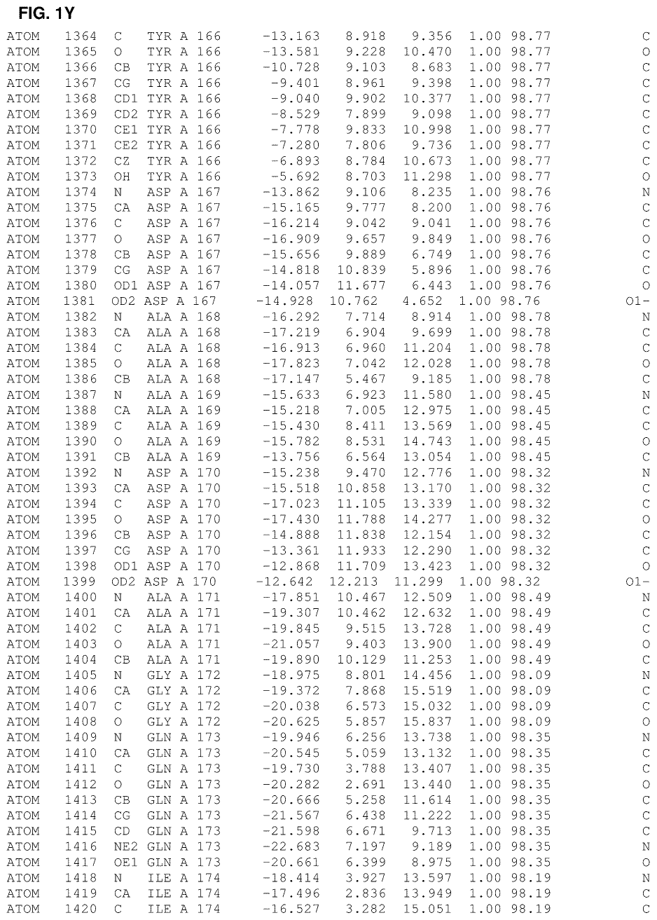

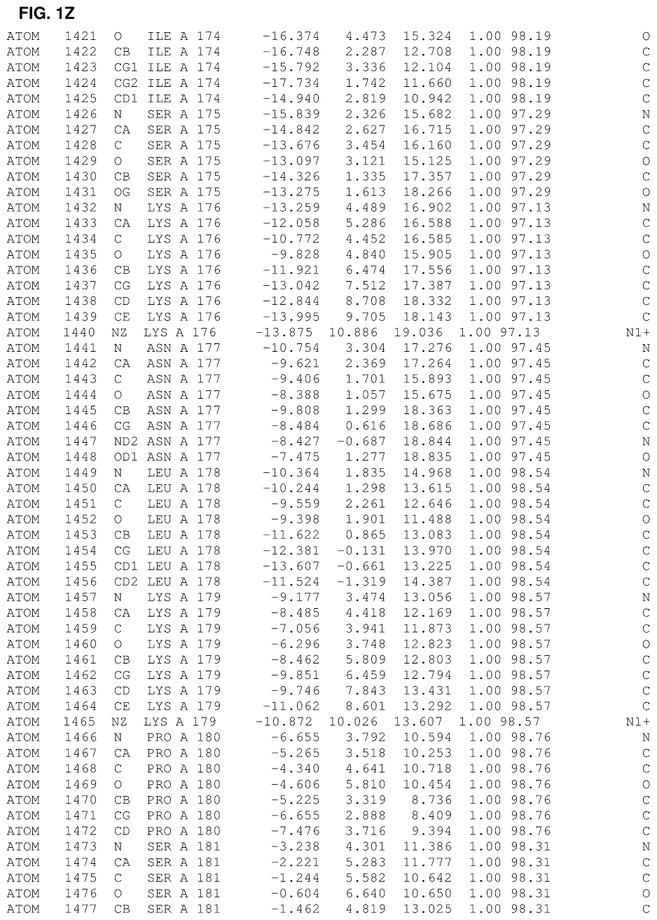

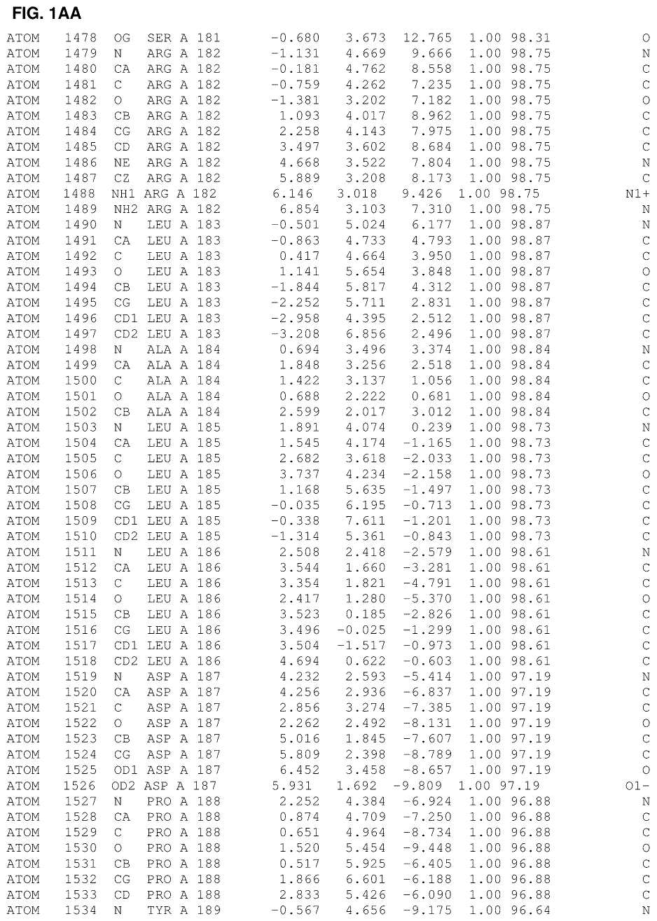

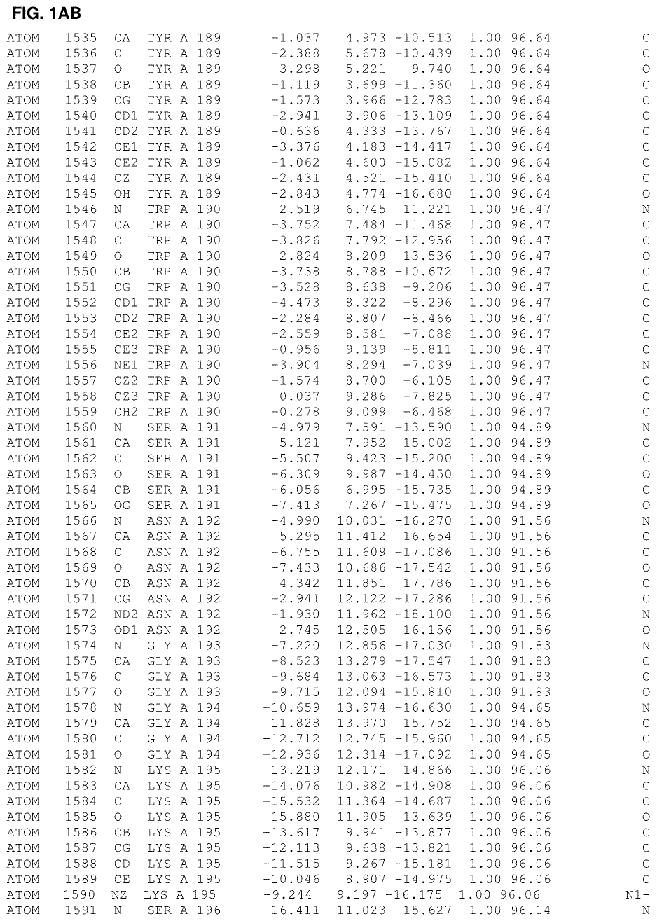

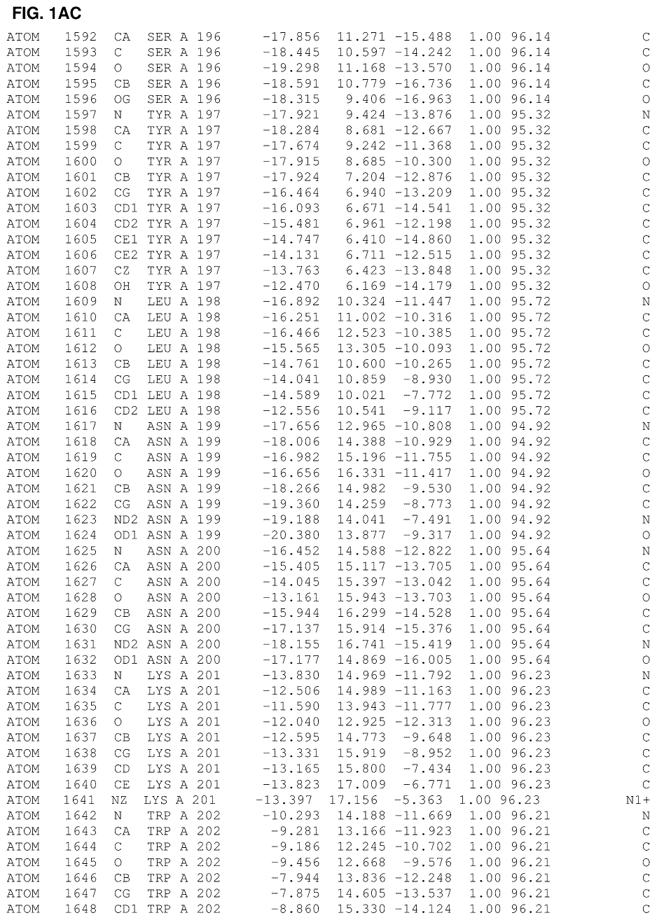

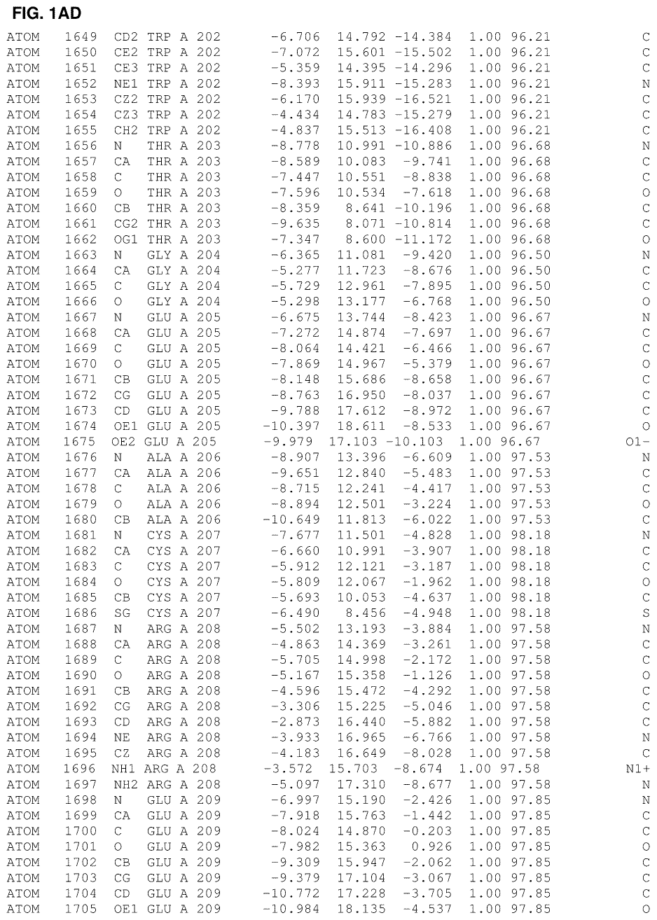

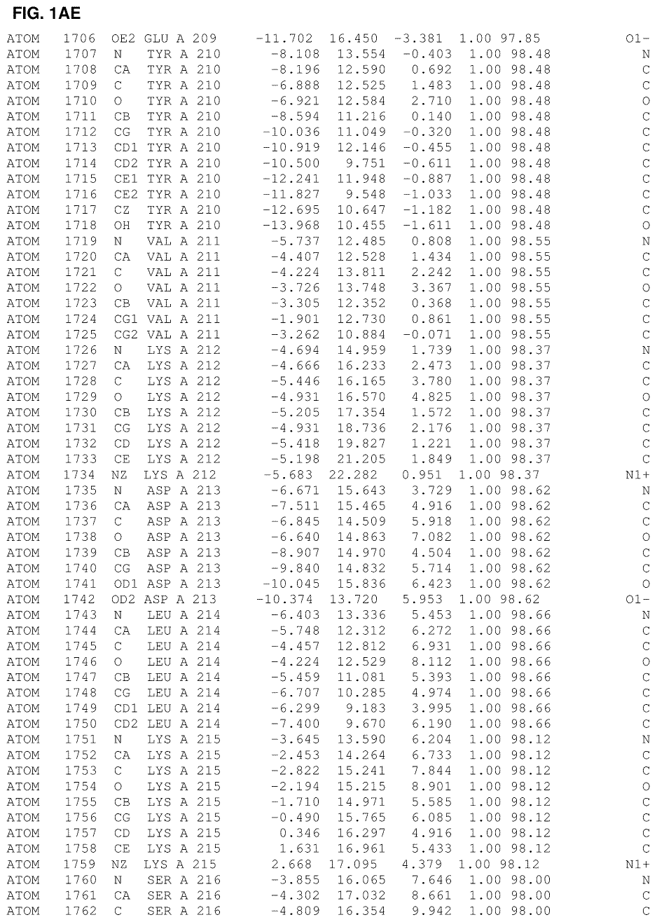

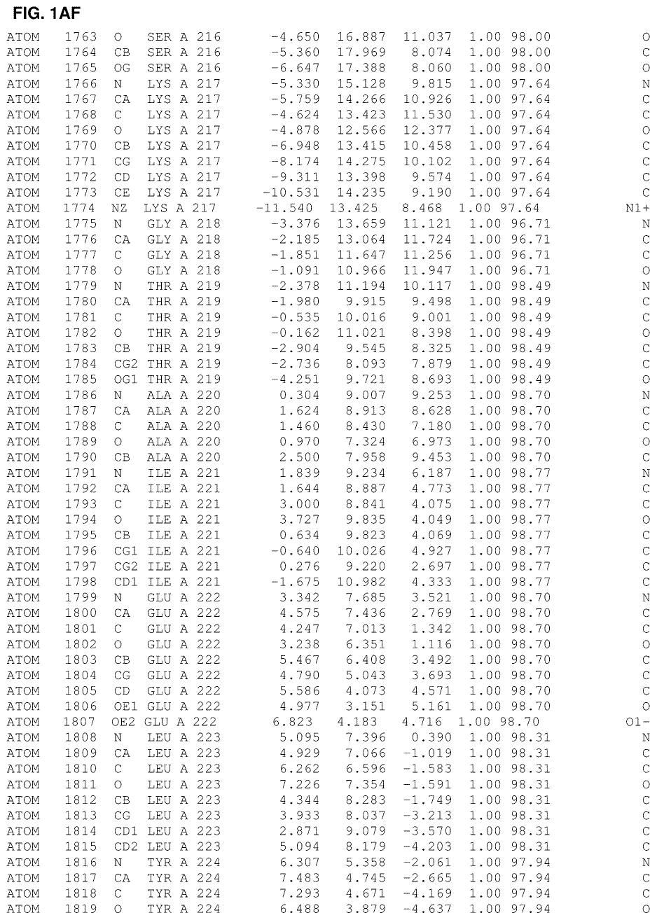

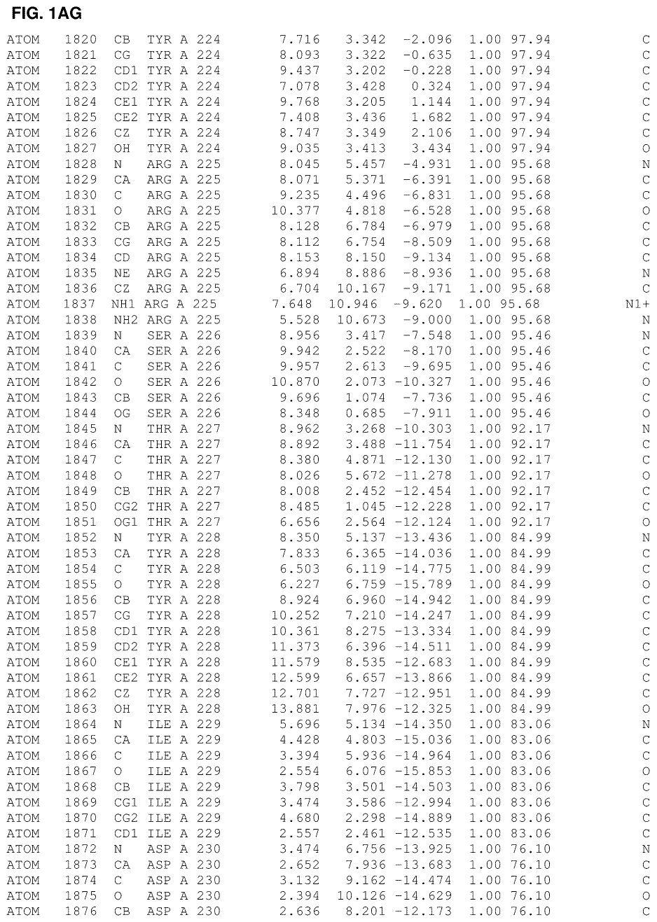

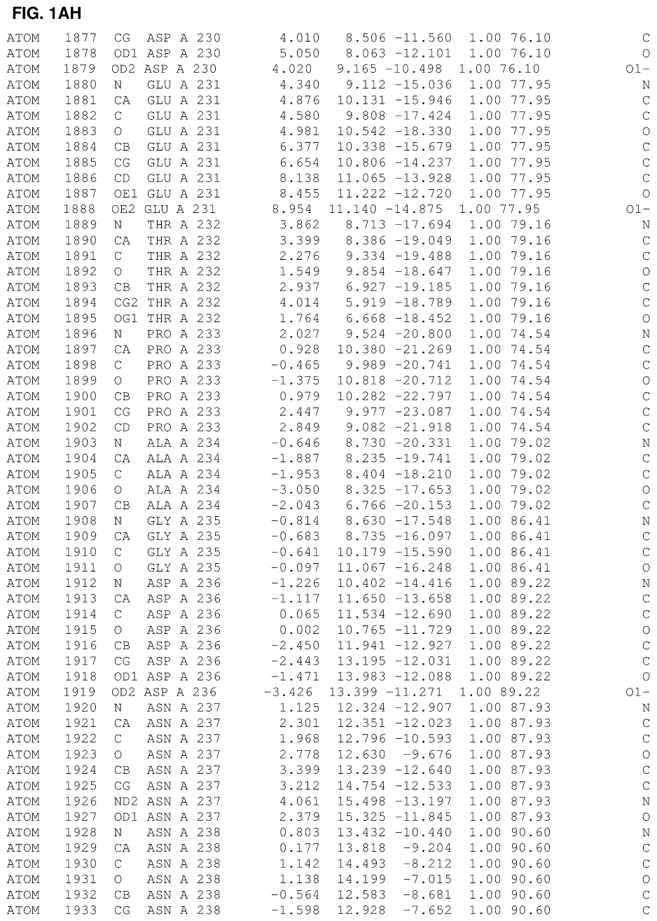

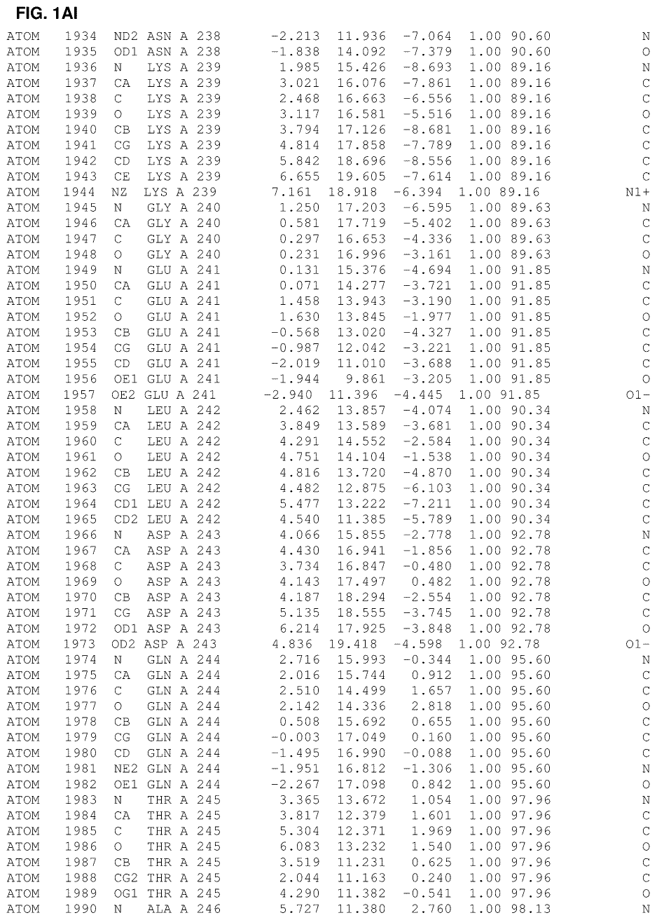

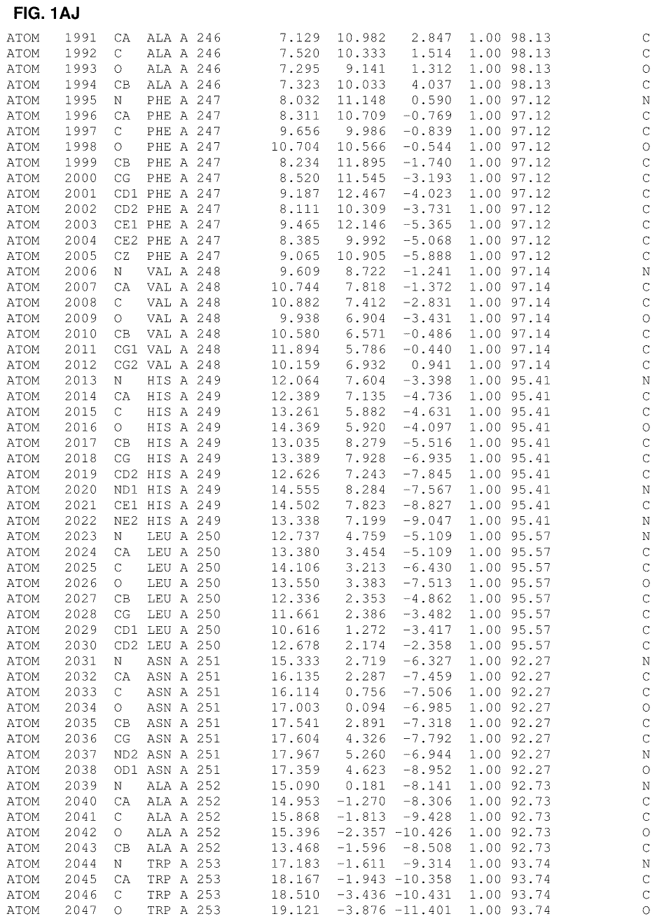

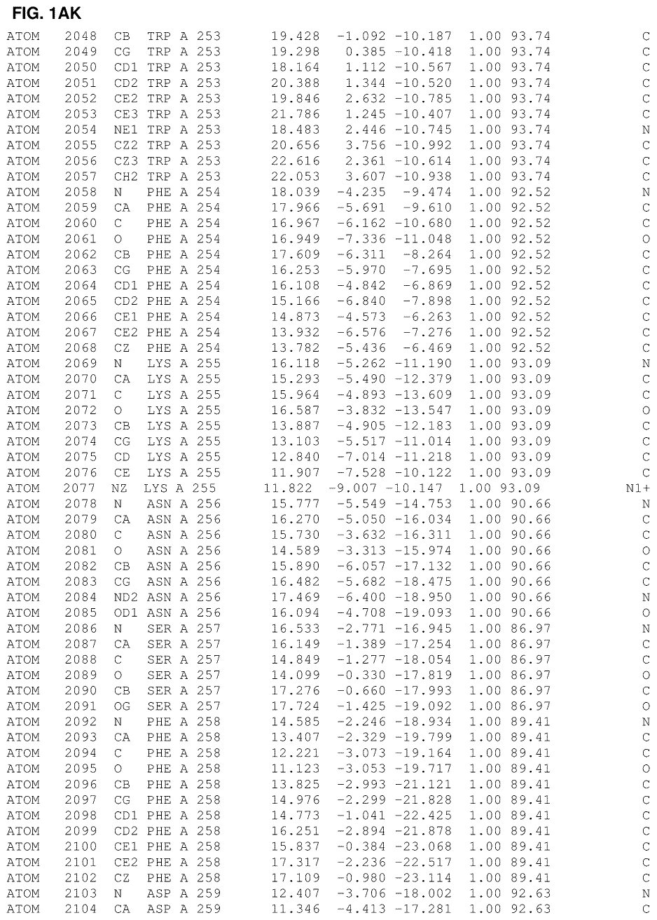

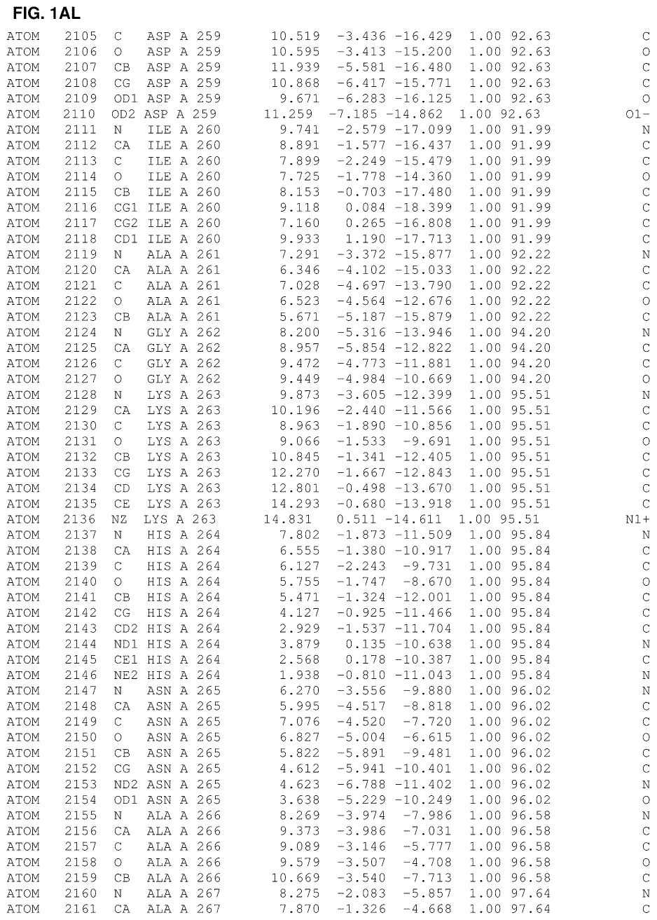

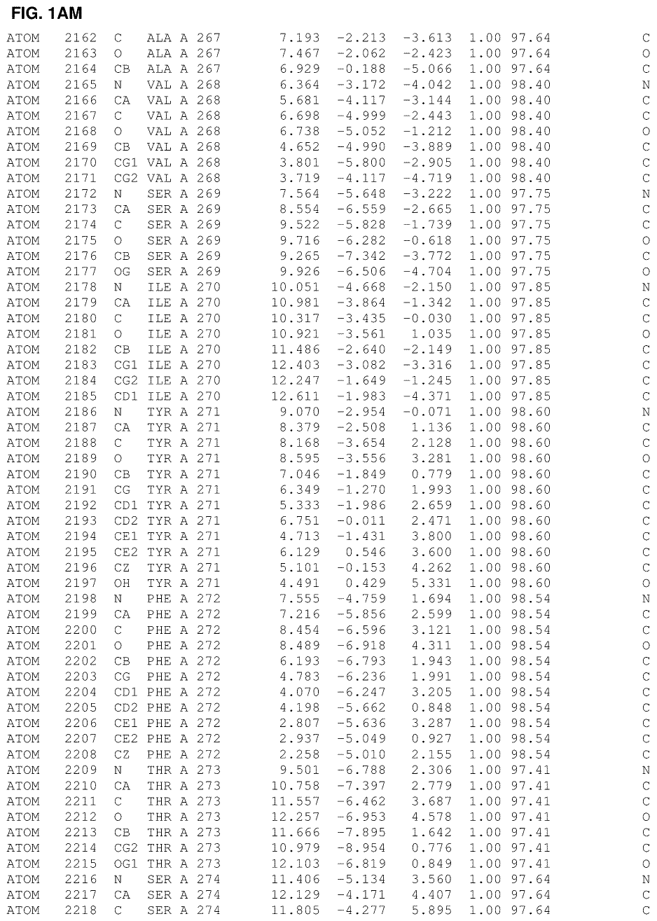

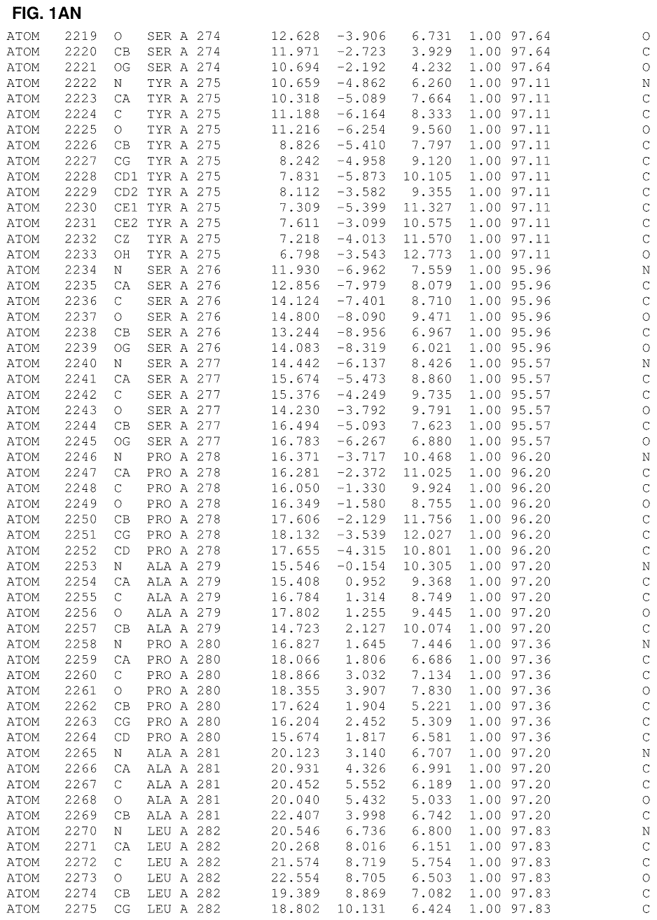

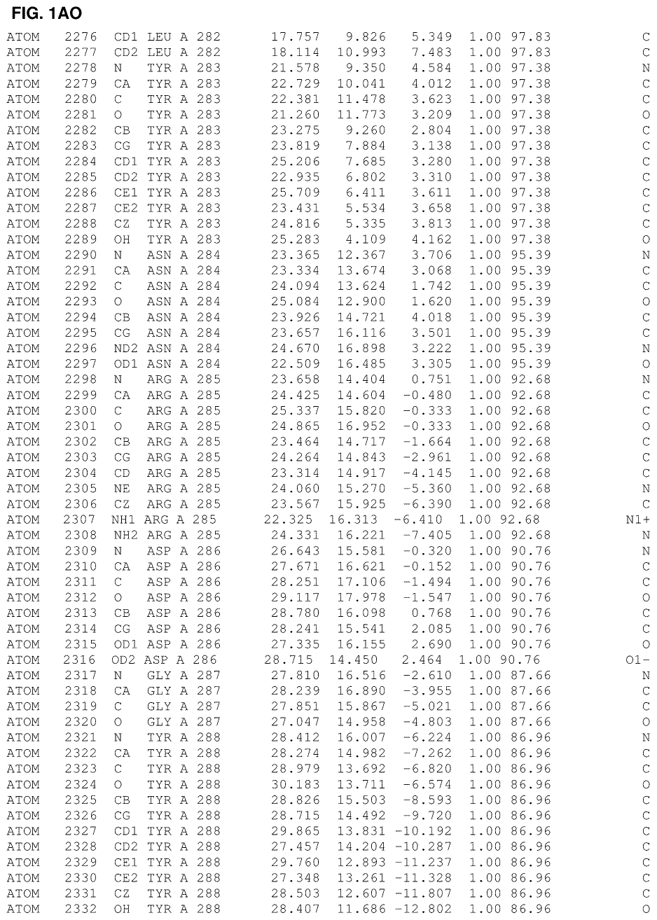

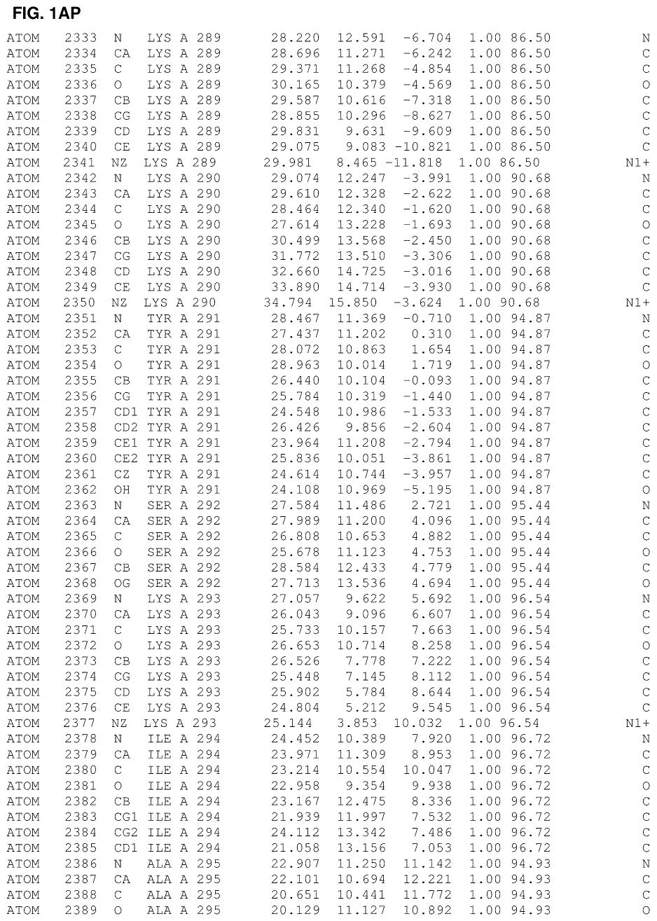

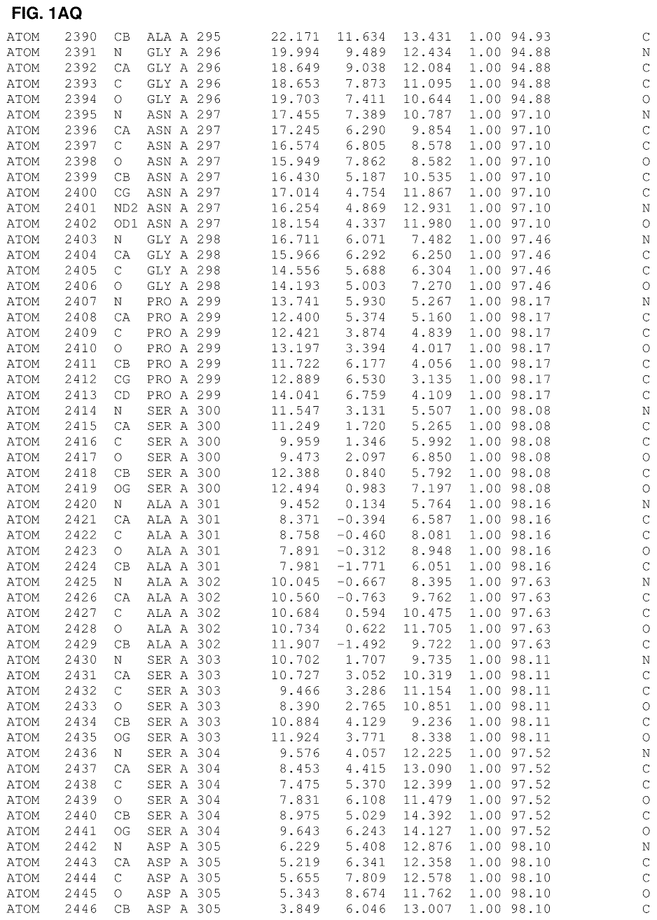

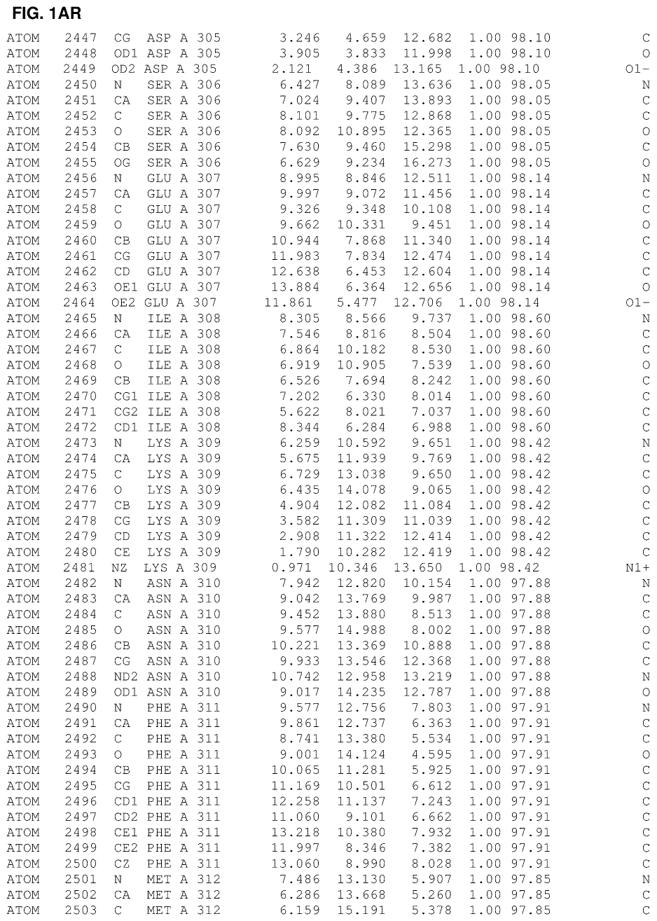

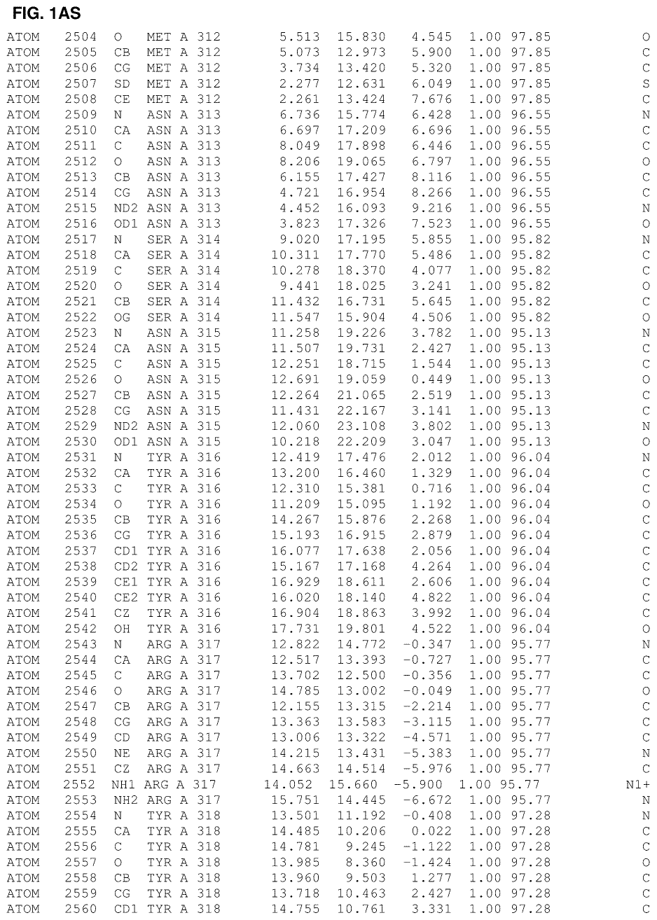

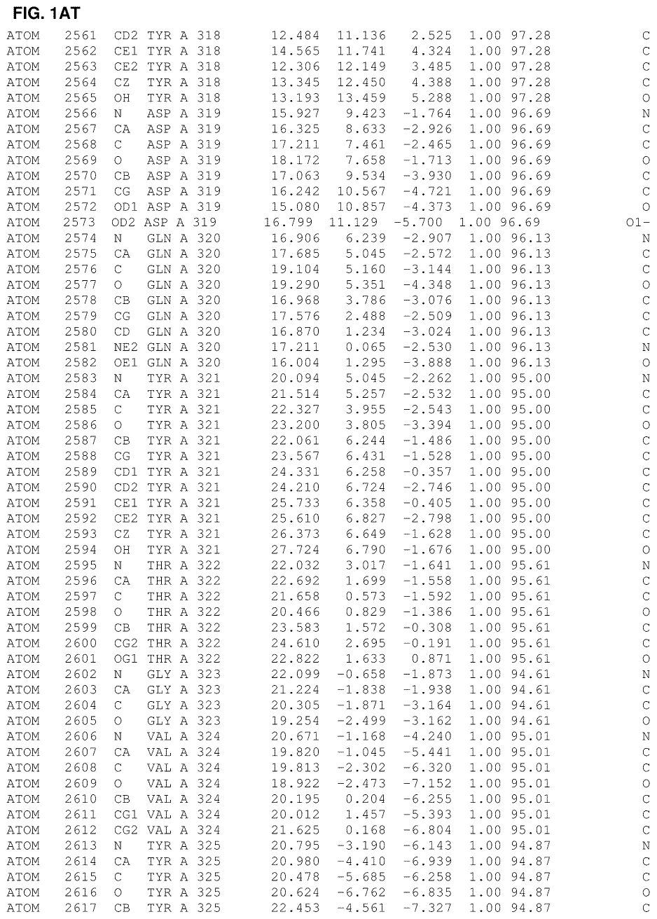

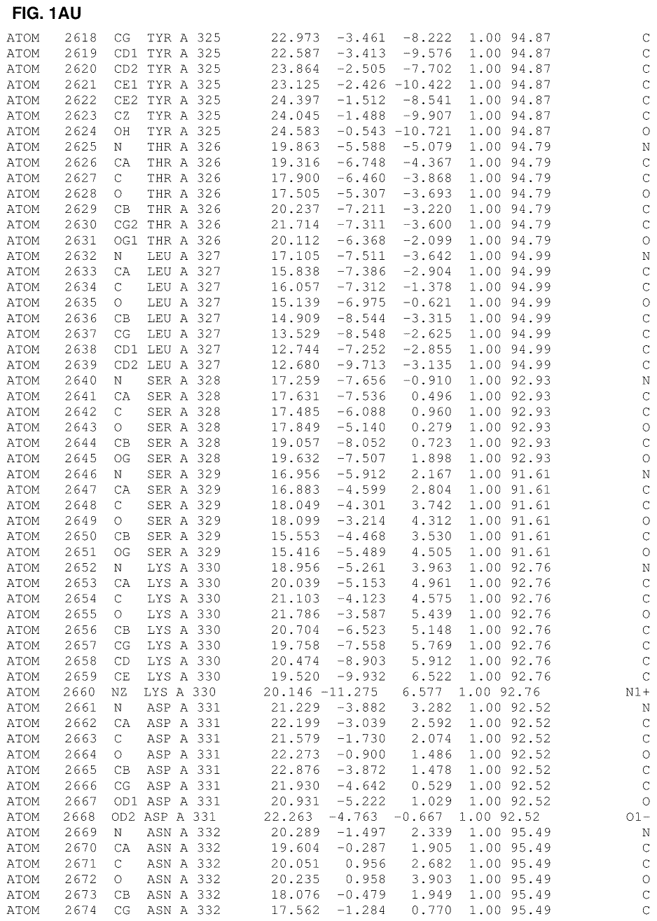

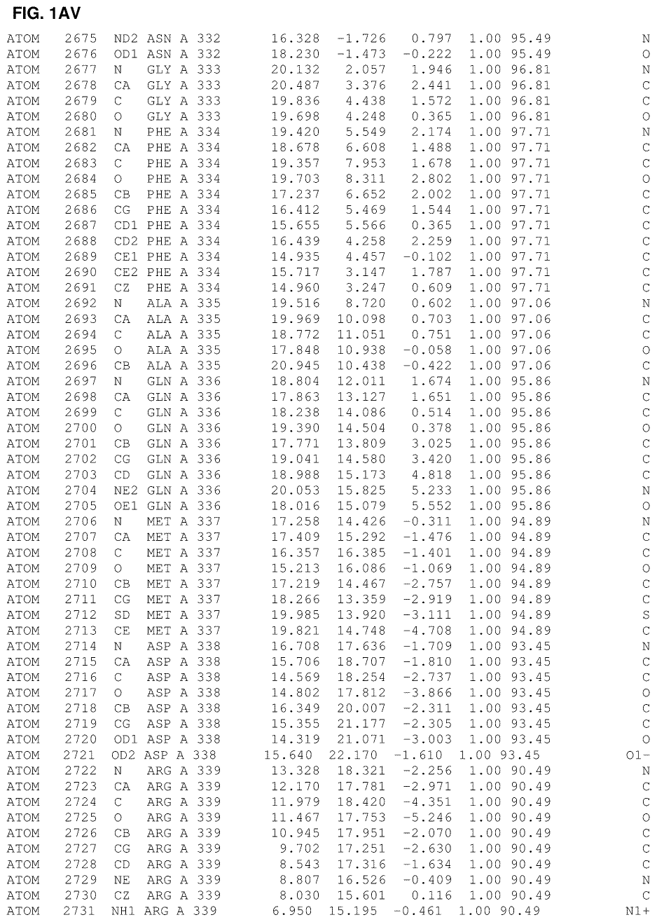

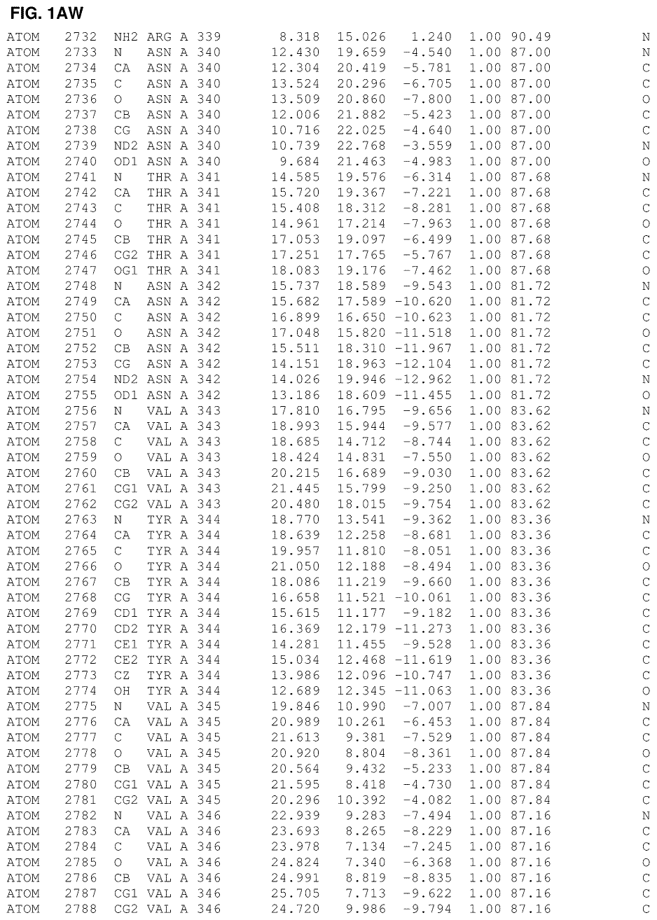

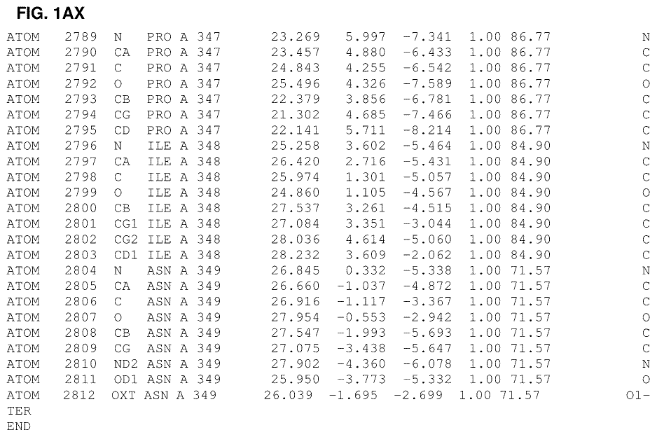

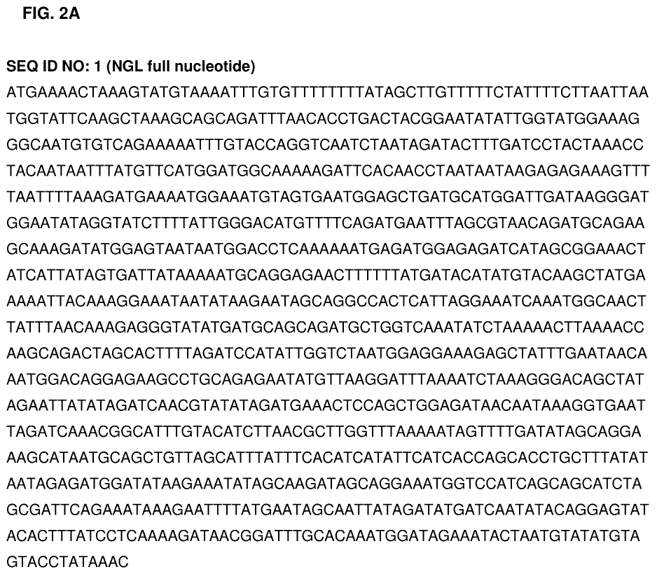

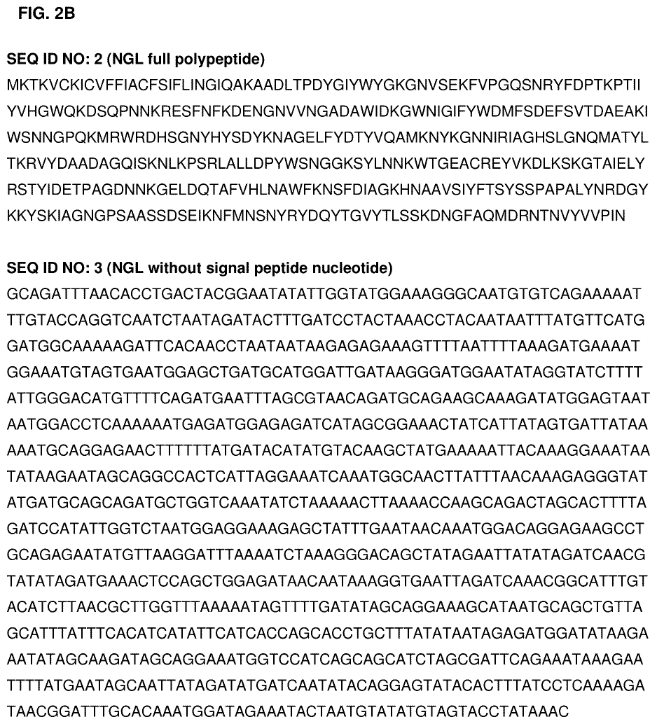

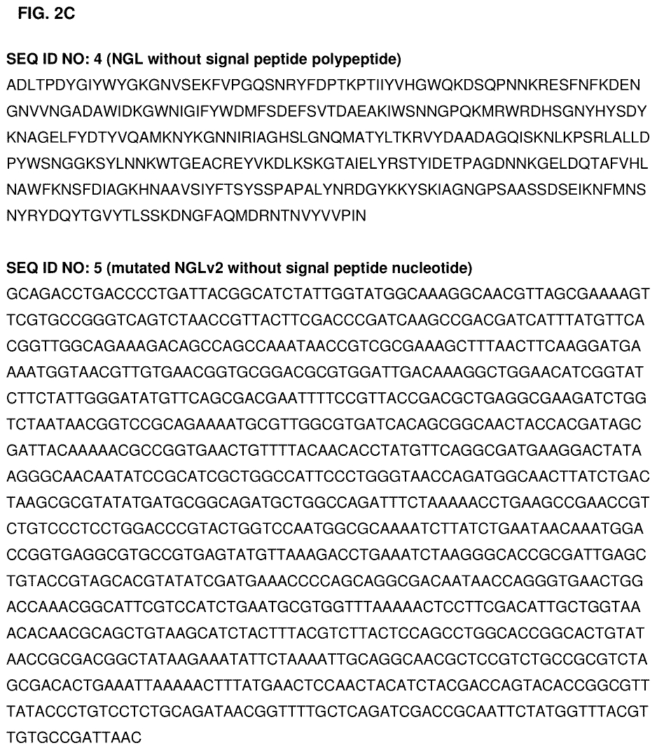

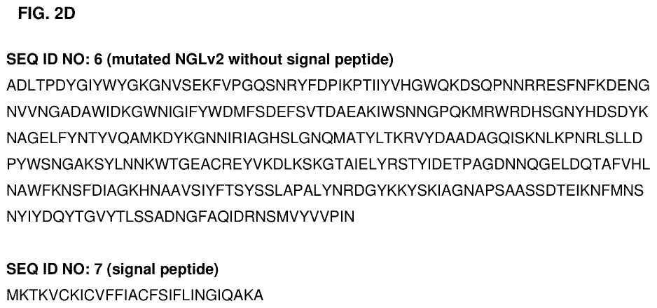

In particular embodiments, the three dimensional protein coordinates of the atoms of the amino acid sequence of the galactolipase (i.e. the polypeptide with galactolipase activity) as taught herein are defined by the set of atomic structural coordinates displayed in FIG. 1 or have a root mean square deviation from the set of atomic structural coordinates displayed in FIG. 1 (i.e. defining atom 1 to atom 2812) of not more than 1.8 Å, not more than 1.7 Å, not more than 1.6 Å, not more than 1.5 Å, not more than 1.0 Å or not more than 0.5 Å. In other words, in particular embodiments, the three ̊dimensional protein coordinates of the atoms of its amino acid sequence are defined by the set of atomic structural coordinates displayed in FIG. 1 or fall within 1.8 Å deviation of the coordinates of the equivalent atoms in FIG. 1 (i.e. defining atom 1 to atom 2812). In further particular embodiments, the galactolipase (i.e. the polypeptide with galactolipase activity) as taught herein has a structure with atomic structural coordinates displayed in FIG. 1 (i.e. defining atom 1 to atom 2812), or with coordinates having a root mean square deviation therefrom, with respect to conserved backbone atoms of the listed amino acid sequence, of not more than not more than 1.8 Å, not more than 1.7 Å, not more than 1.6 Å, not more than 1.5 Å, not more than 1.0 Å or not more than 0.5 Å.

As used herein, “root mean square deviation” is the square root of the arithmetic mean of the squares of the deviations from the mean, and is a way of expressing deviation or variation from the structural coordinates described herein. The present disclosure includes all embodiments comprising conservative substitutions of the noted amino acid residues resulting in same structural coordinates within the stated root mean square deviation.

The three ̊dimensional protein coordinates of the atoms of the amino acid sequence of a polypeptide, protein or peptide may be determined by, for example, but not limited to, X ̊ray diffraction of a crystal of said polypeptide, protein or peptide, or may be predicted using AlphaFold software (v2.3.1 Monomere, available at https://github.com/deepmind/alphafold). Alphafold software takes a protein sequence as input, such as SEQ ID NO: 4. The system extracts various features from the protein sequence, including the identity of the amino acids, their positions in the sequence, and information about their surrounding environment. Alphafold uses a neural network to predict the distance between pairs of amino acids in the protein sequence. Based on the predicted distances, Alphafold predicts the angles between the amino acids. Alphafold then calculates the torsion angles, which describe the rotation of each amino acid around its backbone. Using the predicted torsion angles and other parameters, Alphafold determines the precise coordinates of each atom in the protein. Finally, Alphafold “folds” the protein into a three ̊dimensional structure using the predicted atomic coordinates. The system then uses a second neural network to refine the structure and improve the accuracy of the predicted atomic coordinates.

“Structural coordinates” are the Cartesian coordinates corresponding to an atom's spatial relationship to other atoms in a molecule or molecular complex. Structural coordinates can be obtained by experimental methods like x ̊ray crystallography techniques or NMR techniques, or can be derived by modelisation (homology or de novo) using the protein sequence as input. Various software programs allow for the graphical representation of a set of structural coordinates to obtain a three ̊dimensional representation of a molecule or molecular complex. The structural coordinates of the structures described herein can be modified from the original set provided in FIG. 1 by mathematical manipulation, such as by inversion or integer additions or subtractions. As such, it is recognized that the structural coordinates of the present invention are relative, and are in no way specifically limited by the actual x, y, z coordinates of FIG. 1. In some embodiments, the galactolipase (i.e. the polypeptide with galactolipase activity) as taught herein has at least 90.0%, at least 91.0%, or at least 92.0% structural similarity, preferably at least 93.0%, at least 94.0%, at least 95.0%, at least 96.0%, at least 97.0%, at least 98.0%, at least 99.0% or 100.0% structural similarity with the structure of the polypeptide comprising, consisting essentially of or consisting of the amino acid sequence of SEQ ID NO: 2, SEQ ID NO: 4 or SEQ ID NO: 6, preferably of SEQ ID NO: 2 or SEQ ID NO: 4, more preferably SEQ ID NO: 4.

The structure of a polypeptide, protein or peptide as referred to herein is the (predicted) three ̊dimensional (3D) structure of the polypeptide, protein or peptide. This may encompass the secondary and/or tertiary level of the protein structure.

In the context of the present invention the term “structural similarity” corresponds to the relatedness between two protein structures, defined as their topological similarity. For purposes of the present invention, the structural similarity between two protein structures may be determined using the TM score calculated by the TM ̊align algorithm (Zhang and Skolnick, 2004, PROTEINS: Structure, Function, and Bioinformatics 57:702 ̊710) as implemented in the TM ̊align Debian ̊based package (tm ̊align, described in Zhang and Skolnick, 2005, Nucleic Acid Res 33:2302 ̊2309), preferably version 20190822 or later. The TM ̊score is normalized by the length of the reference protein. In particular the reference protein is SEQ ID NO:2 or SEQ ID NO: 4.

In some embodiments the galactolipase (i.e. the polypeptide with galactolipase activity) as taught herein has a TM ̊score of at least 0.90, more preferably at least 0.91, at least 0.92, at least 0.93, at least 0.94, at least 0.95, at least 0.96, at least 0.97, at least 0.98, at least 0.99 or 1.0 when compared with SEQ ID NO:2 structure or SEQ ID NO: 4 structure (coordinates supplied in FIG. 1.). In other words, a TM score of 0.9 corresponds to a structural similarity of 90.0% and a TM score of 1.0 corresponds to a structural similarity of 100.0%.

The polypeptide with galactolipase activity as taught herein may be obtained from any microorganism of any genus. In particular embodiments, the polypeptide with galactolipase activity as taught herein is a Clostridium galactolipase (i.e. obtained from a strain of Clostridium), such as, but not limited to, a Clostridium saccharobutylicum galactolipase.

The polypeptide with galactolipase activity as taught herein may be obtained and/or identified from other sources of microorganisms or by other means such as, but not limited to, a search in sequences databases for polypeptides predicted to be of the enzyme class with EC number EC 3.1.1.26. or for polypeptides with unknown function having topological/structural/sequences similarity/identity with SEQ ID NO: 2 or SEQ ID NO: 4.

A further aspect provides an isolated nucleic acid capable of encoding or encoding a polypeptide with galactolipase activity as taught herein, preferably comprising a sequence having at least 80.0%, at least 85.0% sequence identity, preferably at least 90.0%, more preferably at least 91.0%, at least 92.0%, at least 93.0%, at least 94.0%, at least 95.0%, at least 96.0%, at least 97.0%, at least 98.0%, at least 99.0% or 100.0% sequence identity to SEQ ID NO:1 (comprising a signal peptide) or SEQ ID NO: 3 (not comprising a signal peptide).

A further aspect provides an expression vector comprising a nucleic acid as taught herein.

The skilled person will understand that if it is envisaged to express and secrete the polypeptide with galactolipase activity as taught herein by a host cell, the nucleic acid encoding the polypeptide with galactolipase activity as taught herein preferably encodes a precursor form of the polypeptide with galactolipase activity including an N ̊terminal signal peptide sequence. Accordingly, the nucleic acid may encode a fragment of the precursor polypeptide of the polypeptide with galactolipase activity as taught herein (i.e. including the signal peptide). In particular embodiments, the nucleic acid encodes a polypeptide with galactolipase activity comprising, consisting essentially of, or consisting of an amino acid sequence as set forth in SEQ ID NO: 4 or SEQ ID NO: 6, preferably SEQ ID NO: 4, or an amino acid sequence having at least 25.0%, at least 30.0%, at least 35.0%, at least 40.0%, at least 45.0%, at least 50.0%, at least 55.0%, at least 60.0%, at least 65.0%, at least 70.0%, at least 75.0%, at least 80.0%, at least 85.0%, at least 90.0%, at least 95.0%, or at least 99.0% sequence identity to SEQ ID NO: 4 or SEQ ID NO: 6, preferably SEQ ID NO: 4, wherein said amino acid sequence is preceded N ̊terminally by a signal peptide having an amino acid sequence MKTKVCKICVFFIACFSIFLINGIQAKA (SEQ ID NO: 7). Alternatively, the nucleic acid encoding the polypeptide with galactolipase activity as taught herein may be comprised within a vector providing for a signal peptide. The signal peptide may be a homologous or heterologous signal peptide, depending on the host cell used for production of the polypeptide with galactolipase activity as taught herein. Furthermore, for prokaryotic expression of the polypeptide with galactolipase activity as taught herein, a protease cleavage site motif may be present C ̊terminally of said signal peptide and N ̊terminally of the polypeptide with galactolipase activity as taught herein.

A further aspect provides a host cell comprising a nucleic acid as taught herein or an expression vector as taught herein, preferably wherein said host cell is a bacterial, fungal or yeast cell.

Further provided herein is the use of the polypeptide with galactolipase activity as taught herein or in preparing a food product.

In particular embodiments, the food product is (a) a dough or a dough product, (b) a batter or a batter product; or (c) a bakery or patisserie product. Further provided herein is the use of the polypeptide with galactolipase activity as taught herein as a food additive. In particular embodiments, the food additive is a bread improver. In other words, the polypeptide with galactolipase activity as taught herein may advantageously be part of a composition, such as a bread improver. “Bread improvers” (also referred to as “dough conditioners” or “dough improvers” or “improving agent” or “flour treatment agent”) are typically added to the dough in order to improve texture, volume, flavour and/or freshness of the baked product as well as to improve machinability and stability of the dough. Typically, a bread improver comprises, consists essentially of or consists of: one or more enzymes (such as e.g. amylases (alpha ̊amylases, beta ̊amylases, glucoamylases, raw starch degrading amylases), xylanases (hemicellulases), cellulases, pectinases, proteases, pectate lyases, oxidases (peroxidases, glucose oxidase, pyranose oxidases, hexose oxidases, Lamino acid oxidases, carbohydrate oxidases, sulfurhydryl oxidases), lipoxygenases, dehydrogenases, laccases, transglutaminases, acyltransferases, protein disulfide isomerases), one or more oxidizing or reducing agents (such as e.g. ascorbic acid, glutathione, cysteine), one or more emulsifiers (such as e.g. diacetyl tartaric acid esters of monoglycerides (DATEM), sodium stearoyl lactylate (SSL), calcium stearoyl lactylate (CSL), glycerol monostearate (GMS), rhamnolipids, lecithins, sucroesters, bile salts), one or more lipid materials (such as e.g. margarine, butter, oil, shortening), one or more vitamins (such as e.g. pantothenic acid and vitamin E), one or more gums, (dried) sourdoughs, and/or one or more sources of fibre (such as e.g. oat fibre).

It is a further aspect of the present invention to provide a method for preparing a food product, preferably a baked product, comprising the step of admixing the polypeptide with galactolipase activity as taught herein with a food ingredient.

In particular embodiments, if the method is a method for preparing a baked product, the method may comprise the step of adding to a dough or the batter the polypeptide with galactolipase activity as taught herein. Preferably, the polypeptide with galactolipase activity as taught herein is added to the dough or batter prior to baking. Preferably, in the method as taught herein, said dough or batter has an improved tolerance.

In particular embodiments, the dough or batter as taught herein has an improved dough tolerance compared to a reference dough or batter, wherein said reference dough or batter is a dough or batter prepared in absence of (i.e. without) the polypeptide with galactolipase activity as taught herein. In further particular embodiments, the dough or batter as taught herein has a dough tolerance which is at least 1.0%, at least 2.0%, at least 3.0%, at least 4.0%, at least 5.0%, at least 10.0%, or at least 15.0% higher than the dough tolerance of a reference dough or batter, wherein said reference dough or batter is a dough or batter prepared in absence of (i.e. without) the polypeptide with galactolipase activity as taught herein.

In line therewith, a further aspect provides the use of the polypeptide with galactolipase activity as taught herein in the preparation of a food product with improved dough tolerance, preferably wherein the dough tolerance is at least 1.0%, at least 2.0%, at least 3.0%, at least 4.0%, at least 5.0%, at least 10.0%, or at least 15.0% higher than the dough tolerance of a reference dough or batter, wherein said reference dough or batter is a dough or batter prepared in absence of (i.e. without) the polypeptide with galactolipase activity as taught herein.

Preferably, the baked product is a bakery or patisserie product. Bakery or patisserie products known in the art include, but are not limited to those selected from the group consisting of bread, soft rolls, bagels, donuts, Danish pastry, hamburger rolls, pizza, pita bread, ciabatta, sponge cakes, cream cakes, pound cakes, muffins, cupcakes, steamed cakes, waffles, brownies, cake donuts, yeast raised donuts, baguettes, rolls, crackers, cookies, pie crusts, rusks and/or other baked products. More preferably, the baked product is a bread, a baguette and/or a roll.

In particular embodiments, the food product comprises wholemeal flour. In further particular embodiments, the food product comprises at least 50.0% (w/w), at least 55.0% (w/w), at least 60.0% (w/w), at least 65.0% (w/w), at least 70.0% (w/w), or at least 75.0% (w/w) of wholemeal flour. Wholemeal flour (i.e. wholegrain flour) is flour made using the whole wheat kernel.

In a further aspect, the present invention relates to the use of the polypeptide with galactolipase activity as taught herein in bakery and patisserie applications. It has been found that the use of the polypeptide with galactolipase activity as taught herein allows the reduction or even the suppression of the use of undesired dough or batter ingredients such as emulsifiers. In particular embodiments, the use as taught herein allows to improve the tolerance of the dough or batter compared to a reference dough or batter, wherein said reference dough or batter is a dough or batter prepared in absence of (i.e. without) the polypeptide with galactolipase activity as taught herein. In the context of the present invention, the “dough or batter tolerance” refers to the capacity of a dough or batter, preferably a bakery dough or batter, to maintain its shape in stress conditions such as a prolonged proofing time or mechanical shocks during or after proofing and to provide, after baking, a baked product with properties (e.g. volume) comparable or even better to a baked product obtained with an unstressed dough.

A further aspect provides a food product (e.g.; bread improver), dough product, batter product or bakery product (e.g. baked product) comprising the polypeptide with galactolipase activity as taught herein.

A further aspect provides a food product, dough product, batter product or bakery product obtained by or obtainable by a use or method as taught herein.

In particular embodiments, said food product is a dough or batter comprising flour, preferably wholemeal flour, and from 10 000 to 100 000 SDGU units/kg flour, from 12 000 to 100 000 SDGU units/kg flour, from 20 000 to 100 000 SDGU units/kg flour, or from 30 000 to 90 000 SDGU units/kg flour of the polypeptide with galactolipase activity as taught herein.

A further aspect provides a method for identifying a polypeptide with galactolipase activity for improving dough tolerance, the method comprising the step of determining whether a polypeptide has at least 90.0%, more preferably at least 91.0%, at least 92.0%, at least 93.0%, at least 94.0%, at least 95.0%, at least 96.0%, at least 97.0%, at least 98.0%, at least 99.0% or 100.0%, structural similarity with the structure of the polypeptide comprising the amino acid sequence of SEQ ID NO: 2 or SEQ ID NO: 4. The structural similarity may be defined as described elsewhere herein.

In particular embodiments, the method comprises a step of comparing the structure of a test polypeptide with the polypeptide comprising the amino acid sequence of SEQ ID NO: 2 or SEQ ID NO: 4 and a step of determining that the test polypeptide is a polypeptide with galactolipase activity for improving dough tolerance when the structure of the test polypeptide has at least 90.0%, more preferably at least 91.0%, at least 92.0%, at least 93.0%, at least 94.0%, at least 95.0%, at least 96.0%, at least 97.0%, at least 98.0%, at least 99.0% or 100.0%, structural similarity with the structure of the polypeptide comprising the amino acid sequence of SEQ ID NO: 2 or SEQ ID NO: 4.

In particular embodiments, the method comprises a step of comparing the structure of a test polypeptide with the structure of the polypeptide comprising the amino acid sequence of SEQ ID NO: 2 or SEQ ID NO: 4 and a step of determining that the test polypeptide is a polypeptide with galactolipase activity for improving dough tolerance when the structure of the test polypeptide has a TM ̊score of at least 0.90, more preferably at least 0.91, at least 0.92, at least 0.93, at least 0.94, at least 0.95, at least 0.96, at least 0.97, at least 0.98, at least 0.99 or 1.0 when compared with SEQ ID NO:2 structure (coordinates supplied in FIG. 1.) or SEQ ID NO: 4 structure.

A further aspect provides a method for identifying a polypeptide with galactolipase activity for improving dough tolerance, the method comprising the step of determining whether the three dimensional protein coordinates of the atoms of the amino acid sequence of a polypeptide have a root mean square deviation from the set of atomic structural coordinates displayed in FIG. 1 of not more than 1.8 Å, not more than 1.7 Å, not more than 1.6 Å, not more than 1.5 Å, not more than 1.0 Å or not more than 0.5 Å. The three ̊dimensional protein coordinates of the atoms of the amino acid sequence of a polypeptide, protein or peptide may be determined as described elsewhere herein.

In particular embodiments, the method comprises a step of comparing the three dimensional protein coordinates of the atoms of the amino acid sequence of a test polypeptide with the set of atomic structural coordinates displayed in FIG. 1 and a step of determining that the test polypeptide is a polypeptide with galactolipase activity for improving dough tolerance when the three ̊dimensional protein coordinates of the atoms of the amino acid sequence of said test polypeptide have a root mean square deviation from the set of atomic structural coordinates displayed in FIG. 1 of not more than 1.8 Å, not more than 1.7 Å, not more than 1.6 Å, not more than 1.5 Å, not more than 1.0 Å or not more than 0.5 Å.

The person skilled in the art will understand that the particular embodiments of the products as taught herein also apply to the methods and uses as taught herein and vice versa.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non ̊limiting examples.

### EXAMPLES

### Example 1: Enzyme Activities Determination

### Lipase

The lipase activity is measured using p ̊nitrophenyl palmitate (pNPP) as substrate. The release of yellow p ̊nitrophenol by the lipase is measured by spectrophotometry at 414 nm. 120 μl of 1 mM pNPP solution (dissolved in 0.05 M Na ̊phosphate buffer at pH 7.5 containing 0.69 M acetone and 0.0049 M Triton X ̊100) are mixed with 60 μl of enzyme sample and incubated at 45° C. for 30 min. The absorbance is measured at 414 nm against a substrate blank in 96° wells microplates.

One lipase milliunit (LmU) is defined as the amount of enzyme needed to release one nanomole (nmole) per minute of p ̊nitrophenol from p ̊nitrophenyl palmitate at 45° C. and pH 7.5.

### Phospholipase

The phospholipase activity is measured using 1,2 ̊Dioleoyl ̊sn ̊glycero ̊3 ̊phospho ̊rac ̊1 ̊glycerol (DOPG) as substrate. The release of free fatty acid by phospholipase is measured by spectrophotometry at 550 nm. 10 μL of 1% DOPG substrate solution (dissolved in 0.1M Na ̊phosphate buffer at pH 7.5 containing 5% of Na ̊deoxycholate) are mixed with 101 μL of enzyme sample and incubated at 30° C. for 15 min. Afterwards, 2 μL of 1M orthophosphoric acid are added to stop the reaction. The released free fatty acids are measured using the kit NEFA ̊HR(2) kit from Fujifilm following the manufacturer instructions (addition of 200 μL of reagent R1 and incubation at 37° C. for 10 min; addition of 100 μL of reagent R2 and incubation at 37° C. for 10 min). A calibration curve is established by use of different concentrations of oleic acid provided in the kit. The absorbance is measured at 550 nm against a blank sample in 96 wells microplate.

One phospholipase activity unit (NefU) is defined as the amount of enzyme needed to release 1 nmol of free fatty acid per minute at 30° C. pH 7.5.

### Galactolipase

The galactolipase activity is measured using digalactosyldiacylglycerol (DGDG) as substrate. The release of free fatty acid by galactolipase is measured by spectrophotometry at 550 nm. 10 μL of 1% DGDG substrate solution (dissolved in 0.1M Na ̊phosphate buffer at pH 7.5 containing 5% of Na ̊deoxycholate) are mixed with 10 μL of enzyme sample and incubated at 30° C. for 15 min. Afterwards, 2 μL of 1M orthophosphoric acid are added to stop the reaction. The released free fatty acids are measured using the kit NEFA ̊HR(2) kit from Fujifilm as described above.

One galactolipase activity unit (SDGU) is defined as the amount of enzyme needed to release 1 nmol of free fatty acid per minute at 30° C. pH 7.5.

### Example 2: Cloning, Expression and Fermentation of Galactolipase as Taught Herein

### Cloning of Galactolipase Enzyme Genes

Based on a bioinformatic study (sequence and structure based approaches) SEQ ID NO: 4 has been predicted to be a possible galactolipase enzyme. This sequence is originating from DNA to proteins translations of the full genomic sequence of Clostridium saccharobutylicum DSM 13864 (GenBank accession number: CP006721.1. (http://www.ncbi.nlm.nih.gov/)).

The DNA sequence coding for the polypeptide with sequence SEQ ID NO: 4 as well as the DNA sequence coding for the polypeptide with sequence SEQ ID NO: 6 (a variant with 17 amino acids substitutions) have been synthesized in order to be expressed into the pET 28a(+) plasmid using standard protocols.

The complete synthesized DNA fragments were subcloned into a pUC19 derivative plasmid. These plasmids were used to transform E. coli DH5α® ultracompetent cells. Purified plasmid preparations made with the Pure Yield Midiprep System (Promega) were digested by using appropriate restriction enzymes to isolate the DNA fragments containing the galactolipase enzymes coding sequences. Those fragments were subcloned into the pET 28a(+) cloning vector (Novagen) and the resulting recombinant plasmids were transformed in E. coli BL21 (DE3) cells (Agilent Technologies). Purified plasmid preparations made with the Pure Yield Midiprep System (Promega) were sequenced by using a ABI3700 DNA sequencer (Applied Biosystems). Sequencing of the inserted fragments was carried out using the universal primers T7 promoter and T7 terminator as well as primers corresponding to internal DNA sequences. The sequences obtained were identical to the expected sequences.

### Cultures of the Recombinant Strains and Production of NGL (Corresponding to SEQ ID NO:2 and NGLv2

(Corresponding to SEQ ID NO:6)) 15 ml of a 5 hours preculture (37° C.) of the E. coli BL21 (DE3) cells carrying the galactolipase enzyme genes were centrifuged at 10000 g for 1 minute and the pellet was resuspended in 500 ml Terrific broth (12 g/l Bacto tryptone (Difco), 24 g/l yeast extract (Difco), 4 ml/I glycerol, 12.54 g/l K₂HPO₄, 2.31 g/l KH₂PO₄) containing 200 μg/ml ampicillin in a 2 liters shake flask. The cultures were incubated at 37° C. and 250 rpm until an absorbance at 550 nm of between 3 and 4 was reached whereupon the expression of the enzyme was induced with 1 mM isopropyl ̊1 ̊thio ̊β ̊galactopyranoside.

### Recovery of NGL and NGLv2.

After 15 hours incubation at 37° C. the cells were harvested by centrifugation at 18 000 g for 30 minutes at 4° C., resuspended in 50 mM BICINE containing 10 mM NaCl, disrupted in a prechilled cell disrupter (Panda 2K, Niro Soavi, GEA Process Engineering Division) at 1500 bars and centrifuged at 40 000 g for 30 minutes. Chromosomal DNA was removed from the crude cell lysates by treatment with 0.2% protamine sulfate (Calbiochem) and centrifugation at 40 000 g for 30 minutes. 25 units of benzonase (Merck, Darmstadt, Germany) were then added to the solution.

The enzyme preparations were clarified by an end filtration on a Millipore POD system with a range of cut ̊off from 0.05 to 1 μm, then concentrated by ultrafiltration on a cross flow filtration system (Sartocon ̊Sartorius) with a cut ̊off of 5 kDa. The concentrated enzyme solutions were filtered on a sterile filtration system, including end filtration of 0.8 and 0.22 μm (absolute filter).

### Example 3: Enzymes Characterization

NGL and NGLv2 were assayed for lipase (LIP), phospholipase (PL) and galactolipase (GL) activities as described in Example 1 and compared to available commercial baking enzymes as listed below with their producer and declared activity:
Noopazyme (Novozymes): lipase
Lipopan Max (Novozymes): lipase
Lipopan F (Novozymes): lipase
Panamore Golden (DSM): lipase
Powerbake 4080 (DuPont/Danisco): glycolipase

TABLE 1

| | | Phospho- | Ga- | | |
|---|---|---|---|---|---|
| | Lipase | lipase | lactolipase | | |
| | (LmU/g | (NfU/g | (SDGU/g | Ratio | Ratio |
| | or ml) | or ml) | or ml) | GL/LIP | GL/PL |
| NGL | 0 | 0 | 13000 | ∞ | ∞ |
| NGLv2 | 0 | 0 | 622 | ∞ | ∞ |
| Noopazyme | 83 | 328 | 2840 | 34.2 | 8.7 |
| Lipopan Max | 437 | 111920 | 46250 | 105.8 | 0.4 |
| Lipopan F | 43572 | 22883 | 363015 | 8.3 | 15.9 |
| Panamore Golden | 13092 | 16142 | 229931 | 17.6 | 14.2 |
| Powerbake 4080 | 227 | 116197 | 1328 | 5.9 | 0.0 |

### Example 4: Wholemeal Flour Pan Breads

Breads doughs were prepared using the ingredients of Table 2. A control test was based on the addition of an emulsifier (MD HP20, Puratos, Belgium), known for its positive effect on dough tolerance.

TABLE 2

| in baker's % | 1 | 2 | 3 | 4 | 5 | 6 |
|---|---|---|---|---|---|---|
| Wholemeal flour (Ceres, Belgium) | 100 | 100 | 100 | 100 | 100 | 100 |
| Water | 67.5 | 67.5 | 67.5 | 67.5 | 67.5 | 67.5 |
| Fresh yeast (Bruggeman, Belgium) | 5 | 5 | 5 | 5 | 5 | 5 |
| Salt | 1.7 | 1.7 | 1.7 | 1.7 | 1.7 | 1.7 |
| Improver* | 3 | 3 | 3 | 3 | 3 | 3 |
| Wheat gluten | 3.5 | 3.5 | 3.5 | 3.5 | 3.5 | 3.5 |
| DATEM (MD HP20, Puratos, Belgium) | | 0.3 | | | | |
| NGL (SDGU units/kg flour) | | | 30000 | 75000 | 90000 | |
| NGLv2 (SDGU units/kg flour) | | | | | | 13600 |

*contains ascorbic acid and enzymes (alpha amylase, xylanase)

### Process

All ingredients were mixed together for 2 min at low speed and 8 min at high speed in a Diosna SP24 mixer. Final dough temperature was 27° C. Doughs were moulded manually (600 g dough pieces) and intermediate proofed at 21° C. for 20 min at 50 ̊55% relative humidity in the Bakery lab. Dough pieces (600 g each) were made up using the on the line Quality & Ethics—Jac Unic set at R6/L16 and moulded. Dough pieces to which no shock was applied obtained a final proof at 35° C. for 50 min at 95% relative humidity in a Koma SunRiser. Dough pieces to which shock was applied had a final proof at 35° C. for 70 min at 95% relative humidity in a Koma SunRiser. The shock was applied by letting the baking pans drop from a height of 5.5 cm. Then, doughs were baked at 230° C. during 35 min in a Miwe Condo Oven 2 (Michael Wenz ̊Arnstein—Germany) with steam injection (0,1 L before and 0.2 L after ovening of the breads). Baked breads were cooled down during 180 min before measuring volume.

### Bread Analysis

The volume of the breads was measured using the commonly used rapeseed displacement method. Average volume of four breads was determined. The results are given in Table 3. Values are expressed as the percentage of volume increase/decrease compared to the reference without shock.

TABLE 3
volume increase/decrease compared to reference without shock (1)

| % | 1 | 2 | 3 | 4 | 5 | 6 |
|---|---|---|---|---|---|---|
| Without shock | 0 | 17 | 21 | 17 | 15 | 18 |
| With shock | °14 | 29 | 26 | 29 | 25 | 47 |

### Example 5: Wholemeal Flour Pan Bread

Breads were prepared and evaluated as described in Example 4 using the ingredients listed in Table 4.

TABLE 4

| in baker's % (except NGL) | 1 | 2 | 3 | 4 | 5 | 6 | 7 | 8 |
|---|---|---|---|---|---|---|---|---|
| Wholemeal flour (Ceres, Belgium) | 100 | 100 | 100 | 100 | 100 | 100 | 100 | 100 |
| Water | 67.5 | 67.5 | 67.5 | 67.5 | 67.5 | 67.5 | 67.5 | 67.5 |
| Fresh yeast (Bruggeman, Belgium) | 5 | 5 | 5 | 5 | 5 | 5 | 5 | 5 |
| Salt | 1.7 | 1.7 | 1.7 | 1.7 | 1.7 | 1.7 | 1.7 | 1.7 |
| Improver* | 3 | 3 | 3 | 3 | 3 | 3 | 3 | 3 |
| Wheat gluten | 3.5 | 3.5 | 3.5 | 3.5 | 3.5 | 3.5 | 3.5 | 3.5 |
| DATEM (MD HP20, Puratos, Belgium) | | 0.3 | | | | | | |
| NGL (units/kg flour) | | | 45000 | | | | | |
| Noopazyme | | | | 0.015 | | | | |
| Lipopan max | | | | | 0.015 | | | |
| Lipopan F | | | | | | 0.015 | | |
| Panamore Golden | | | | | | | 0.002 | |
| Powerbake 4080 | | | | | | | | 0.005 |

*contains ascorbic acid and enzymes (alpha amylase, xylanase)

### Evaluation

TABLE 5
volume increase/decrease compared to reference without shock

| | 1 | 2 | 3 | 4 | 5 | 6 | 7 | 8 |
|---|---|---|---|---|---|---|---|---|
| Without shock | 0 | 18 | 15 | 6 | 3 | °1 | 9 | 12 |
| With shock | °14 | 24 | 29 | 9 | 2 | 5 | 13 | 1 |

### Example 6: Croissants

Croissants were prepared using the ingredients of Table 6 for the dough and Aristo croissant (Puratos, Belgium) as lamination fat.

TABLE 6

| in baker's % | 1 | 2 |
|---|---|---|
| Wheat flour (Ceres, Belgium) | 100 | 100 |
| Water | 51 | 51 |
| Fresh yeast (Bruggeman, Belgium) | 6.5 | 6.5 |
| Salt | 1.7 | 1.7 |
| Sugar (sucrose) | 10 | 10 |
| Improver | 2 | 2 |
| Fat (Aristo croissant, Puratos, Belgium) | 25 | 25 |
| DATEM (MD HP20, Puratos, Belgium) | 0.3 | |
| NGL (units/kg flour) | | 60000 |

* contains ascorbic acid and enzymes (alpha amylase, xylanase)

### Process

All ingredients except the lamination fat were mixed together for 4 min at low speed and 3 min at high speed in a Diosna SP24 mixer to obtain dough. Final dough temperature was 20° C. Dough was cooled down during 4 min at ̊18C.° to relax the dough. A first lamination was performed with the lamination fat (2 single folds) followed by a cooling step at ̊18C.° during 30 min in a KOMA freezer. Second lamination was done with the lamination fat (1 single fold) following by cooling in a KOMA freezer during 25 min at ̊18° C. Laminated dough pieces (60 g each) were molded and rolled out. A final proof was performed at 28° C. for 90 min at 80% relative humidity in a Koma proofing box. After proofing, dough pieces were frozen in a Koma blast freezer during 35 min at ̊30° C. until reaching a core temperature of ca. ̊7° C. Frozen croissant doughs were stored during 3 months at ̊18° C. Croissant doughs were defrosted during 30 min at room temperature before final bake ̊off in a Miwe Roll ̊In Oven during 19 min with the following temperature profile: 225° C. during 1 min with steam (0.7 L)—195° C. during 13 min by heating. Baked croissants were cooled down to room temperature and their volume was measured using a 3D scanner.

### Evaluation

Total volume of 5 croissants made with DATEM (croissant “1”) was 1775 ml. Total volume of 5 croissants made with an enzyme as taught herein (croissant “2”) was 1900 ml.

### Example 7: Parbaked Fully Frozen Baguettes

Parbaked fully frozen baguettes using the dough compositions of Table 7.

TABLE 7

| in baker's % | 1 | 2 |
|---|---|---|
| Wheat flour (Ceres, Belgium) | 100 | 100 |
| Water | 57 | 57 |
| Fresh Yeast (Bruggeman, Belgium) | 3 | 3 |
| Salt | 1.7 | 1.7 |
| Improver* | 0.5 | 0.5 |
| NGL (SGDU units/kg flour) | | 20000 |

*contains ascorbic acid and enzymes (alpha amylase, xylanase)

### Process

All ingredients were mixed together in 2 steps: 2 min at low speed and 7 min at high speed in a Diosna SP24 mixer. Final dough temperature was 26° C. Then, dough was rested during 5 min at 25° C. Afterwards, dough pieces (160 g each) were formed and got an intermediate proof during 10 min at 25° C. Molding was performed with a Bertrand T4,5/L3 (+/ ̊28 cm) and followed by a final proof during 75 ̊90 min at 28° C. in a Koma until dough pieces reached a height of 6.5 cm. Then, 3 cuts were scored on each dough piece. First (pre)baking was done in a Miwe Roll In Oven with the following temperature profile: 190° C. during 1 min with steam (1 L)—175° C. during 15 min. The baked products were cooled until 25° C. during 30 ̊40 min and frozen in a Koma blast freezer during 45 ̊60 min at ̊30° C. until a core temperature of ca. 8° C. Prebaked baguettes were stored during 3 weeks at ̊18° C. in a Koma freezer. Before the final baking, the baguettes were defrosted during 40 min at 25° C. Baking was conducted in a Miwe Econo Oven during 8 min at 180° C. Baked baguettes were cooled down to room temperature and their volume was measured using the commonly used rapeseed displacement method.

### Evaluation

The volume of the baguettes made with the enzyme as taught (baguette “2”) herein was 8% higher than the volume of the control baguettes without enzyme (baguette “1”).

### Example 8: Crusty Rolls

Crusty rolls were prepared using the dough compositions of Table 8.

TABLE 8

| Ingredients (baker's %) | 1 | 2 | 3 |
|---|---|---|---|
| Wheat flour | 100 | 100 | 100 |
| Water | 60 | 60 | 60 |
| Fresh Yeast (Bruggeman, Belgium) | 4 | 4 | 4 |
| Sodium Chloride | 1.7 | 1.7 | 1.7 |
| Improver* | 3 | 3 | 3 |
| DATEM (MD HP20, Puratos, Belgium) | | 0.3 | |
| NGL (SGDU units/kg flour) | | | 20000 |

*contains ascorbic acid and enzymes (alpha amylase, xylanase)

### Process

All ingredients were mixed together for 2 min at low speed and 5 min at high speed in an Eberhardt N24 mixer. Final dough temperature as well as the resting and proofing temperatures were 25° C. After resting for 15 min at 25° C., dough was reworked manually and rested for another 10 min. Afterwards, 2 kg sized dough pieces were made up and proofed for 10 min. The 2 kg dough pieces were divided and made up using the Rotamat. Round dough pieces (50 g each) were obtained. After another 5 min resting time, dough pieces were cut by pressing and one half of the dough pieces were submitted to a final proofing stage at 35° C. for 120 min whereas another half of the dough pieces were submitted to a shock test (the tray containing the doughs was dropped on the shelf from a height of 10 cm) and submitted to a final proofing stage at 35° C. for 120 min. Dough pieces were baked at 230° C. in a MIWE Roll ̊In oven with steam (Michael Wenz ̊Arnstein ̊Germany). The volume of 6 rolls was measured using the commonly used rapeseed displacement method.

The results are shown in Table 9.

TABLE 9
Volume increase/decrease compared to the
volume of reference 1 without shock

| | 1 | 2 | 3 |
|---|---|---|---|
| With shock | °18 | 4 | 9 |

### Example 9: Modelization of the SEQ ID NO: 4 Structure

Alphafold (https://github.com/deepmind/alphafold) has been used to predict the structure of SEQ ID NO: 4, using the protein sequence as input. Default parameters of the algorithm have been used (CASP14 model). The generated output is a file with atoms coordinates as depicted in FIG. 1.

### Example 10: Use of SEQ ID NO: 4 Structure (FIG. 1) to Identify Similar Structures

The tool Foldseek (available at https://github.com/steineggerlab/foldseek) is used to calculate TM ̊scores between the SEQ ID NO: 4 structure (FIG. 1) versus a database containing all structures available on the Alphafold Protein structure Database (available at https://alphafold.ebi.ac.uk/), with e ̊value=0.001, alignment ̊type: TM ̊align, mode=easy ̊search). The use of Foldseek allows to compute TM ̊scores and root mean square deviations as described in Zhang and Skolnick, 2004 (PROTEINS: Structure, Function, and Bioinformatics 57:702 ̊710) between SEQ ID NO:4 structure and the 200 millions of structures from the Alphafold Protein structure Database without the requirement of a super ̊computer. Structures with a TM ̊score value from 0.9 to 1 (RMSD from 0 to 1.8 Å) are considered as homologous galactolipase to SEQ ID NO: 4 structure.

1. (canceled)

2. The method according to claim 12, wherein the ratio of galactolipase activity to phospholipase activity is 1000.0 or higher.

3. The method according to claim 12, wherein the ratio of galactolipase activity to lipase activity is 300.0 or higher.

4. The method according to claim 12, wherein the polypeptide with galactolipase activity is selected from:
(a) a polypeptide comprising a sequence having at least 80.0% sequence identity to the amino acid sequence of SEQ ID NO: 2 or SEQ ID NO: 4;
(b) a polypeptide comprising a sequence encoded by a polynucleotide comprising a sequence having at least 80.0% sequence identity to the nucleotide sequence of SEQ ID NO:1 or SEQ ID NO: 3;
(c) a fragment of the polypeptide of (a) or (b) having galactolipase activity, or;
(d) a polypeptide having at least 90.0% structural similarity with the structure of the polypeptide comprising the amino acid sequence of SEQ ID NO: 2 or SEQ ID NO: 4.

5. (canceled)

6. The method according to claim 12, wherein the polypeptide with galactolipase activity is a Clostridium galactolipase.

7. (canceled)

8. The method according to claim 12, wherein the food product is (a) a dough or a dough product, (b) a batter or a batter product; or (c) a bakery or patisserie product.

9. The method according to claim 12, wherein the food ingredient is a bread improver.

10. (canceled)

11. The method according to claim 13, wherein the admixing of the polypeptide with galactolipase activity with the dough or batter increases the tolerance of the dough or a batter as compared to an otherwise identical dough or batter lacking the polypeptide with galactolipase activity.

12. A method for preparing a food product, the method comprising:
admixing a polypeptide with galactolipase activity with a food ingredient;
wherein the polypeptide with galactolipase activity has:
a ratio of galactolipase activity to phospholipase activity of 100.0 or higher,
and/or a ratio galactolipase activity to lipase activity of 150.0 or higher.

13. The method according to claim 12, wherein the food product is a bakery or patisserie product and the polypeptide with galactolipase activity is admixed with a dough or batter.

14. The method according to claim 12, wherein the food product further comprises wholemeal flour.

15. (canceled)

16. An expression vector comprising a nucleic acid encoding a polypeptide with galactolipase activity and a sequence heterologous to the nucleic acid sequence;
wherein the polypeptide with galactolipase activity comprises
a ratio of galactolipase activity to phospholipase activity of 100.0 or higher,
and/or a ratio galactolipase activity to lipase activity of 150.0 or higher.

17. (canceled)

18. The expression vector of claim 16, wherein the expression vector is comprised in a host cell.

19. A method for identifying a polypeptide with galactolipase activity for improving dough tolerance, the method comprising the step of determining whether a polypeptide has at least 90.0% structural similarity with the structure of the polypeptide comprising the amino acid sequence of SEQ ID NO: 2 or SEQ ID NO: 4.

20. (canceled)


SEQUENCE LISTING (7 sequences)

SEQ ID NO: 1 - DNA, 1131 bp, Clostridium saccharobutylicum
  source 1..1131: mol_type = unassigned DNA
atgaaaactaaagtatgtaaaatttgtgttttttttatagcttgtttttctattttcttaattaatggtattcaagctaaagcagcagatttaacacctgactacggaatatattggtatggaaagggcaatgtgtcagaaaaatttgtaccaggtcaatctaatagatactttgatcctactaaacctacaataatttatgttcatggatggcaaaaagattcacaacctaataataagagagaaagttttaattttaaagatgaaaatggaaatgtagtgaatggagctgatgcatggattgataagggatggaatataggtatcttttattgggacatgttttcagatgaatttagcgtaacagatgcagaagcaaagatatggagtaataatggacctcaaaaaatgagatggagagatcatagcggaaactatcattatagtgattataaaaatgcaggagaacttttttatgatacatatgtacaagctatgaaaaattacaaaggaaataatataagaatagcaggccactcattaggaaatcaaatggcaacttatttaacaaagagggtatatgatgcagcagatgctggtcaaatatctaaaaacttaaaaccaagcagactagcacttttagatccatattggtctaatggaggaaagagctatttgaataacaaatggacaggagaagcctgcagagaatatgttaaggatttaaaatctaaagggacagctatagaattatatagatcaacgtatatagatgaaactccagctggagataacaataaaggtgaattagatcaaacggcatttgtacatcttaacgcttggtttaaaaatagttttgatatagcaggaaagcataatgcagctgttagcatttatttcacatcatattcatcaccagcacctgctttatataatagagatggatataagaaatatagcaagatagcaggaaatggtccatcagcagcatctagcgattcagaaataaagaattttatgaatagcaattatagatatgatcaatatacaggagtatacactttatcctcaaaagataacggatttgcacaaatggatagaaatactaatgtatatgtagtacctataaac

SEQ ID NO: 2 - protein, 377 aa, Clostridium saccharobutylicum
MKTKVCKICVFFIACFSIFLINGIQAKAADLTPDYGIYWYGKGNVSEKFVPGQSNRYFDPTKPTIIYVHGWQKDSQPNNKRESFNFKDENGNVVNGADAWIDKGWNIGIFYWDMFSDEFSVTDAEAKIWSNNGPQKMRWRDHSGNYHYSDYKNAGELFYDTYVQAMKNYKGNNIRIAGHSLGNQMATYLTKRVYDAADAGQISKNLKPSRLALLDPYWSNGGKSYLNNKWTGEACREYVKDLKSKGTAIELYRSTYIDETPAGDNNKGELDQTAFVHLNAWFKNSFDIAGKHNAAVSIYFTSYSSPAPALYNRDGYKKYSKIAGNGPSAASSDSEIKNFMNSNYRYDQYTGVYTLSSKDNGFAQMDRNTNVYVVPIN

SEQ ID NO: 3 - DNA, 1047 bp, Clostridium saccharobutylicum
  source 1..1047: mol_type = unassigned DNA
gcagatttaacacctgactacggaatatattggtatggaaagggcaatgtgtcagaaaaatttgtaccaggtcaatctaatagatactttgatcctactaaacctacaataatttatgttcatggatggcaaaaagattcacaacctaataataagagagaaagttttaattttaaagatgaaaatggaaatgtagtgaatggagctgatgcatggattgataagggatggaatataggtatcttttattgggacatgttttcagatgaatttagcgtaacagatgcagaagcaaagatatggagtaataatggacctcaaaaaatgagatggagagatcatagcggaaactatcattatagtgattataaaaatgcaggagaacttttttatgatacatatgtacaagctatgaaaaattacaaaggaaataatataagaatagcaggccactcattaggaaatcaaatggcaacttatttaacaaagagggtatatgatgcagcagatgctggtcaaatatctaaaaacttaaaaccaagcagactagcacttttagatccatattggtctaatggaggaaagagctatttgaataacaaatggacaggagaagcctgcagagaatatgttaaggatttaaaatctaaagggacagctatagaattatatagatcaacgtatatagatgaaactccagctggagataacaataaaggtgaattagatcaaacggcatttgtacatcttaacgcttggtttaaaaatagttttgatatagcaggaaagcataatgcagctgttagcatttatttcacatcatattcatcaccagcacctgctttatataatagagatggatataagaaatatagcaagatagcaggaaatggtccatcagcagcatctagcgattcagaaataaagaattttatgaatagcaattatagatatgatcaatatacaggagtatacactttatcctcaaaagataacggatttgcacaaatggatagaaatactaatgtatatgtagtacctataaac

SEQ ID NO: 4 - protein, 349 aa, Clostridium saccharobutylicum
ADLTPDYGIYWYGKGNVSEKFVPGQSNRYFDPTKPTIIYVHGWQKDSQPNNKRESFNFKDENGNVVNGADAWIDKGWNIGIFYWDMFSDEFSVTDAEAKIWSNNGPQKMRWRDHSGNYHYSDYKNAGELFYDTYVQAMKNYKGNNIRIAGHSLGNQMATYLTKRVYDAADAGQISKNLKPSRLALLDPYWSNGGKSYLNNKWTGEACREYVKDLKSKGTAIELYRSTYIDETPAGDNNKGELDQTAFVHLNAWFKNSFDIAGKHNAAVSIYFTSYSSPAPALYNRDGYKKYSKIAGNGPSAASSDSEIKNFMNSNYRYDQYTGVYTLSSKDNGFAQMDRNTNVYVVPIN

SEQ ID NO: 5 - DNA, 1047 bp, synthetic construct
  source 1..1047: mol_type = other DNA
gcagacctgacccctgattacggcatctattggtatggcaaaggcaacgttagcgaaaagttcgtgccgggtcagtctaaccgttacttcgacccgatcaagccgacgatcatttatgttcacggttggcagaaagacagccagccaaataaccgtcgcgaaagctttaacttcaaggatgaaaatggtaacgttgtgaacggtgcggacgcgtggattgacaaaggctggaacatcggtatcttctattgggatatgttcagcgacgaattttccgttaccgacgctgaggcgaagatctggtctaataacggtccgcagaaaatgcgttggcgtgatcacagcggcaactaccacgatagcgattacaaaaacgccggtgaactgttttacaacacctatgttcaggcgatgaaggactataagggcaacaatatccgcatcgctggccattccctgggtaaccagatggcaacttatctgactaagcgcgtatatgatgcggcagatgctggccagatttctaaaaacctgaagccgaaccgtctgtccctcctggacccgtactggtccaatggcgcaaaatcttatctgaataacaaatggaccggtgaggcgtgccgtgagtatgttaaagacctgaaatctaagggcaccgcgattgagctgtaccgtagcacgtatatcgatgaaaccccagcaggcgacaataaccagggtgaactggaccaaacggcattcgtccatctgaatgcgtggtttaaaaactccttcgacattgctggtaaacacaacgcagctgtaagcatctactttacgtcttactccagcctggcaccggcactgtataaccgcgacggctataagaaatattctaaaattgcaggcaacgctccgtctgccgcgtctagcgacactgaaattaaaaactttatgaactccaactacatctacgaccagtacaccggcgtttataccctgtcctctgcagataacggttttgctcagatcgaccgcaattctatggtttacgttgtgccgattaac

SEQ ID NO: 6 - protein, 349 aa, synthetic construct
ADLTPDYGIYWYGKGNVSEKFVPGQSNRYFDPIKPTIIYVHGWQKDSQPNNRRESFNFKDENGNVVNGADAWIDKGWNIGIFYWDMFSDEFSVTDAEAKIWSNNGPQKMRWRDHSGNYHDSDYKNAGELFYNTYVQAMKDYKGNNIRIAGHSLGNQMATYLTKRVYDAADAGQISKNLKPNRLSLLDPYWSNGAKSYLNNKWTGEACREYVKDLKSKGTAIELYRSTYIDETPAGDNNQGELDQTAFVHLNAWFKNSFDIAGKHNAAVSIYFTSYSSLAPALYNRDGYKKYSKIAGNAPSAASSDTEIKNFMNSNYIYDQYTGVYTLSSADNGFAQIDRNSMVYVVPIN

SEQ ID NO: 7 - protein, 28 aa, synthetic construct
MKTKVCKICVFFIACFSIFLINGIQAKA